SEQUENCE LISTING (13 sequences)

SEQ ID NO: 1 - protein, 138 aa, Artificial Sequence
  CD79 alpha in construct
LLMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQ

SEQ ID NO: 2 - protein, 157 aa, Artificial Sequence
  CD79 beta in construct
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDD

SEQ ID NO: 3 - protein, 111 aa, Artificial Sequence
  CD3 zeta in construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPP

SEQ ID NO: 4 - protein, 41 aa, Artificial Sequence
  CD28 in construct
RSKRSRLLHSDYMNMTPRRPGPTRKHYQPYAPPRDFAAYRS

SEQ ID NO: 5 - protein, 683 aa, Artificial Sequence
  Total construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 41 aa, Artificial Sequence
  4-1BB
RGRKKLLYIFKQPFMRPVQTTQEEDGCSCRFPEEEEGGCEL

SEQ ID NO: 7 - protein, 194 aa, Artificial Sequence
  CD79alpha wildtype
LWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 8 - protein, 200 aa, Artificial Sequence
  CD79beta wildtype total
ARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDKAGMEEDHTYEGLDIDQTATYEDIVTLRTGEVKWSVGEHPGQE

SEQ ID NO: 9 - protein, 683 aa, Artificial Sequence
  CD79-4-1BB-CD3zeta construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 683 aa, Artificial Sequence
  CD79-4-1BB-CD28-CD3zeta construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 476 aa, Artificial Sequence
  CD 79 wildtype construct
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKPGSGATNFSLLKQAGDVEENPGPMARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDKAGMEEDHTYEGLDIDQTATYEDIVTLRTGEVKWSVGEHPGQE

SEQ ID NO: 12 - protein, 796 aa, Artificial Sequence
  CD19 construct
MEFGLSWLFLVAILKGVQCEVKLQESGPGLVAPSQSLSVTCTVSGVSLPDYGVSWIRQPPRKCLEWLGVIWGSETTYYNSALKSRLTIIKDNSKSQVFLKMNSLQTDDTAIYYCAKHYYYGGSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGAGSGATNFSLLKQAGDVEENPGPMDMRVPAQLLGLLLLWLPGARCDIQMTQTTSSLSASLGDRVTISCRASQDISKYLNWYQQKPDGTVKLLIYHTSRLHSCVPSRFSGSGSGTDYSLTISNLEQEDIATYFCQQGNTLPYTFGGGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 796 aa, Artificial Sequence
  CD20 construct
MEFGLSWLFLVAILKGVQCQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGAGSGATNFSLLKQAGDVEENPGPMDMRVPAQLLGLLLLWLPGARCQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2017/0354681 A1
(43) Pub. Date: Dec. 14, 2017

(54) T CELL-BASED IMMUNOTHERAPEUTICS

(71) Applicant: BCRT Holding BV, Utrecht (NL)

(72) Inventors: Mark De Boer, Blaricum (NL); Carsten Linnemann, Amsterdam (NL); Antonius Nicolaas Maria Schumacher, Amsterdam (NL); Riccardo Mezzadra, Amsterdam (NL)

(73) Assignee: BCRT Holding BV, Utrecht (NL)

(21) Appl. No.: 15/518,358

(22) PCT Filed: Oct. 26, 2015
(86) PCT No.: PCT/EP2015/074752

§ 371 Date: Apr. 11, 2017

(30) Foreign Application Priority Data
Oct. 24, 2014 (EP) 14190210.6

(51) Int. Cl. A61K 35/17; C12N 5/0783; C07K 16/28; C07K 14/705; A61K 48/00; A61K 35/12

(52) U.S. Cl. CPC A61K 35/17; C07K 16/2803; C07K 16/2887; C07K 14/70503; C12N 5/0636; A61K 2035/124; C07K 2319/03; C07K 2319/02; C12N 2510/00; A61K 48/00

(57) ABSTRACT

The present invention provides compositions and methods for immunotherapy in human. The invention includes a B cell receptor like complex expressed in T cells and comprising an extracellular antigen recognition domain, a trans-membrane domain, a CD79αβ heterodimer, and a signaling region that controls T cell activation. The extracellular antigen recognition domain and trans-membrane are derived from the same human or humanized B cell receptor and form a single unit in the complex. The signaling region comprises a T cell signaling domain in combination with a co-stimulatory signaling domain. The signaling region is fused to the CD79αβ heterodimer. Furthermore, the B cell receptor like complex of the present invention can use a targeting molecule as a bridge between cytotoxic T cells and targeted cells.

### FIELD OF THE INVENTION

The invention relates to T cell-based immunotherapeutics and methods of using the therapeutics in immunotherapy, such as in the treatment of cancer.

### BACKGROUND TO THE INVENTION

The immune system is designed to eradicate a large number of pathogens with minimal immunopathology to non-infected tissues. Immunotherapy is an emerging treatment modality that seeks to harness the power of the human immune system to treat diseases, in particular cancer. One immunotherapy method for enhancing the cellular immune response in subjects is a type of cell therapy called adoptive cell transfer (ACT). ACT is a cell therapy that involves the removal of immune cells from a subject, the ex vivo manipulation (i.e. activation, purification and/or expansion of the cells) and the subsequent infusion of the resulting cell product back into the same subject. Adoptive T cell therapies represent a potent treatment modality for cancer exploring the capacity of CD8⁺ T cells to recognize and destroy malignant cells, which present peptides derived from tumor-associated antigens. However, adoptive T cell therapies generally rely on the availability of pre-existing tumor-reactive CD8⁺ T cells within the patient.

In an attempt to overcome the dependency on pre-existing tumor-reactive T cells in cancer patients, gene therapies have been developed which aim to introduce genes coding for tumor-reactive receptors into patient-derived T cells. Receptors utilized for such gene therapies include conventional TCRαβ and TCRγδ genes but also “designer” receptors that allow targeting of structures normally not recognized by T cells, such as defined tumor cell surface antigens. Targeting antigens at the tumor surface becomes possible by fusion of an antigen-binding moiety, most commonly the single-chain variable fragments (scFv) from the antigen-binding sites of a monoclonal antibody, together with a trans-membrane domain and a T-cell activating domain. This artificial immune receptor is expressed at the surface of T cells and will trigger T-cell effector functions upon binding of the antigen-binding domain to its target antigen. Nowadays, these types of artificial lymphocyte signaling receptors are commonly referred to as chimeric antigen receptors (CARs).

Chimeric antigen receptor T-cells (CAR-T cells) have shown very promising clinical benefits for certain cancer patients. A typical CAR-T cell construct consists of an ecto-domain consisting of the heavy chain (V_{H}) and light chain (V_{L}) domains of an anti-tumor target antibody in scFv format, fused to a flexible trans-membrane domain such as derived from CD8 or CD28, fused to an endo-domain consisting of an activation domain of a co-stimulatory molecule such as 4-1BB and fused to tyrosine-based activation motif such as that from CD3 (Sadelain et al. Cancer Discovery 2013; 3(4): 388-398). T cells expressing such a construct can recognize and destroy cancer cells expressing the tumor-associated antigen in an MHC-independent manner.

Recently, a multi-chain CAR concept was introduced wherein the signaling domains in juxtamembrane position are present on polypeptide(s) distinct from that carrying the extracellular ligand binding domain (WO2014039523). Although this multi-chain CAR concept provides a more flexible architecture for CARs, its extracellular ligand binding domain is still chimeric containing fusion sites of different proteins. Despite impressive pre-clinical and early clinical studies in patients with both solid tumors and hematopoietic malignancies (Sadelain et al. Cancer Discovery 2013; 3(4): 388-398), there are currently a number of limitations hampering the generalized clinical application of CAR-T cells and there remain several challenges to overcome in order to achieve significant clinical benefits.

Most importantly, currently used CAR-designs have shown to be immunogenic. This immunogenicity is potentially driven by two independent components: 1) the use of an extracellular antigen recognition domain that is not fully human or humanized and 2) the fusion of protein domains derived from different proteins. This can introduce unwanted immune responses that can jeopardize the therapeutic effects, e.g. by impeding the persistence of CAR-modified T cells. It is well known that fusion of two different proteins can create so called neo-epitopes. These neo-epitopes can lead to unwanted immune reactions (Sadelain et al Cancer Discovery 2013; 3(4): 388-398). It is also well documented with the use of chimeric (mouse/human) therapeutic antibody drugs that patients can react to the mouse-derived sequences and generate a so-called human anti-mouse antibody (HAMA) response (Sadelain et al., Cancer Discovery 2013; 3(4): 388-398; Maus et al. Cancer Immunol Res 2013; 1(1): 26-31). This, on top of limiting the therapeutic benefit, can generate symptoms similar to an allergic reaction that ranges from a mild rash to life-threatening complications. Due to these known disadvantages of CAR-T cells, researchers have continuously been working to modify the CAR-T cells to improve CAR-T cell function and reduce the side effects, but so far, the above-mentioned problems have not been solved yet.

One of the solutions to recognize target cells expressing target-associated antigens in an MHC-independent manner and without the disadvantages of the CAR-T cells is the use of an immunoglobulin (Ig) antigen receptor (also called B cell receptor) in T cells. In Costa et al. (J. Exp. Med. 1992; 175: 1669-1676) the Ig antigen receptor of B lymphocytes was functionally reconstituted in the Jurkat T cell line by transfection in the presence of B29 (CD79 beta). In a corresponding patent application (WO9318161) a DNA construct comprising the expression sequences, fragments or derivatives thereof, which code for both an IgM immunoglobulin and the B29 protein was disclosed. These reports only indicate that transport of IgM to the surface of T cells required co-expression of the Ig heavy and light chains with CD79 beta. Further, minimal activation of the IgM receptor was shown, but only in the Jurkat T cell line and only after direct incubation with monoclonal antibodies and not after contact with target cells.

Thus, there is a clear need in the art for improved compositions and methods for immunotherapy using human or humanized T-cell associated antibody constructs, without extracellular fusion sites that can result in immunogenic neo-epitopes. The present invention addresses this unmet need.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY OF THE INVENTION

The present invention is directed to compositions and methods for treatment of diseases, including but not limited to cancer, using a human or humanized B cell receptor like complex system that controls T cell activation. More in particular, the B cell receptor like complex comprises an extracellular antigen recognition and trans-membrane domain from a human or humanized B cell receptor protein in combination with a CD79 protein or a functional equivalent thereof and a signaling region. This signaling region comprises a T cell receptor signaling domain in combination with a co-stimulatory domain. Typical for this invention, the signaling region is fused to the CD79 protein. The B cell receptor like complex can work in concert with many different tumor-targeting molecules. Because the extracellular antigen recognition domain and the trans-membrane domain are derived from the same human or humanized B cell receptor protein and additionally form a single unit in the complex, extracellular fusion sites are not present in the ecto-domain of the receptor and hence, unwanted and hazardous immunogenicity/immune responses are avoided with these constructs during antibody mediated immune recognition. In addition, the chimeric part of this complex is only situated intracellular at the site where the CD79 protein and the signaling region are fused together.

The present invention provides an isolated B cell receptor like complex, i.e. an isolated B cell receptor like protein, comprising an extracellular antigen recognition domain, a trans-membrane domain, a CD79 protein or functional equivalent thereof, and a signaling region that controls T cell activation. The extracellular antigen recognition domain and the trans-membrane domain are derived from the same human or humanized B cell receptor protein and form a single human or humanized B cell receptor protein in the complex. Typical for this invention, the human or humanized B cell receptor protein is combined with a CD79 protein and a signaling region. The signaling region comprises a T cell signaling domain and a co-stimulatory domain. Also typical for this invention, the signaling region is fused to the CD79 protein. The CD79 protein as used herein may consist of a CD79α protein (SEQ ID NO.: 1), a CD79β protein (SEQ ID NO.: 2), a CD79α homodimer, a CD79β homodimer, a CD79αβ heterodimer, or any functional equivalent thereof. In one embodiment the CD79 protein consists of a CD79α protein or any functional equivalent thereof; in particular a CD79α protein. In another embodiment the CD79 protein consists of a CD79β protein or any functional equivalent thereof; in particular a CD79β protein. In another embodiment the CD79 protein consists of a CD79αβ heterodimer or any functional equivalent thereof; in particular a CD79αβ heterodimer.

The invention further provides one or more isolated nucleic acid sequences encoding a B cell receptor like complex, wherein the B cell receptor like complex comprises an extracellular antigen recognition domain, a trans-membrane domain, a CD79 protein or a functional equivalent thereof, and a signaling region that controls T cell activation. The extracellular antigen recognition domain and the trans-membrane domain are derived from the same human or humanized B cell receptor protein and form a single unit in the complex. The signaling region comprises a T cell signaling domain in combination with a co-stimulatory domain, wherein the signaling region is fused to the CD79 protein. The CD79 protein as used herein may consist of a CD79α protein, a CD79β protein, a CD79α homodimer, a CD79β homodimer, a CD79αβ heterodimer, or any functional equivalent thereof, including the different embodiments as mentioned hereinbefore.

In one embodiment of the present invention, the signaling region is fused to one or both monomers of the CD79 protein or functional equivalent thereof. In one embodiment the T cell signaling domain and the co-stimulatory domain are fused to one another thereby composing the signaling region. In an even further embodiment said fused T cell signaling domain, the co-stimulatory domain or both are further fused to one or both monomers of the CD79 protein.

Within the different embodiments of the present invention, the extracellular antigen recognition domain and trans-membrane domain form a single unit in the complex. In a particular embodiment of the present invention, the extracellular antigen recognition domain of the B cell receptor protein within the complex binds to a surface antigen. In another embodiment, the extracellular antigen recognition domain of the B cell receptor like complex binds to a universal epitope expressed on a targeting molecule.

In one embodiment of the present invention, the targeting molecule is a protein scaffold and in another embodiment the targeting molecule is selected from the group consisting of scFv molecules, Darpin molecules, Nanobody molecules, Alphabody molecules, Centyrin molecules, Affibody molecules, heavy chain only antibodies or molecules from any other protein scaffold platform. In one embodiment, the targeting molecule binds to a surface antigen. In another particular embodiment, the surface antigen is associated with a solid or hematologic tumor.

Another aspect of the present invention is based on the concept that the surface antigen, to which the extracellular antigen recognition domain of the B cell receptor is directed, is an antigenic substance or a combination of substances produced in tumor cells that trigger an immune response in the host. Known tumor antigens include but are not limited to CD19, CD20, CD22, HER1, HER2, HER3, ROR1, mesothelin, CD33/IL3Ra, c-Met, PSMA, Glycolipid F77, EGFRvIII, GD-2, NY-ESO-1 TCR, and MAGE-A3 TCR.

Typical for this invention, the human or humanized B cell receptor is combined with the CD79 protein or functional equivalent thereof and a signaling region, in which the signaling region is fused to one or both monomers of the CD79 protein as present. In one embodiment, the signaling region comprises a T cell signaling domain in combination with a co-stimulatory domain. The T cell signaling domain contains one or multiple ITAM motifs leading to T cell activation. In a particular embodiment, the T cell signaling domain is selected from the group of molecules consisting of CD3 zeta, CD3 epsilon, CD3 delta, CD3 gamma, and other CD3 like sequences. In one embodiment the T cell signaling domain consists of a CD3 zeta domain or any functional equivalent thereof; in particular a CD3 zeta domain (SEQ ID NO.: 3). In another embodiment, the T cell signaling domain consists of a CD3 epsilon domain or any functional equivalent thereof; in particular a CD3 epsilon domain. In another embodiment, the T cell signaling domain consists of a CD3 delta domain or any functional equivalent thereof; in particular a CD3 delta domain. In another embodiment, the T cell signaling domain consists of a CD3 gamma domain or any functional equivalent thereof; in particular a CD3 gamma domain. In another embodiment, the co-stimulatory signaling region comprises one or more fragments of the intracellular domain of a co-stimulatory molecule selected from the group consisting of, but not limited to, CD27, CD28, 4-1BB, OX40, CD30, CD40L, ICOS, lymphocyte function-associated antigen-1 (LFA-1), CD2, CD7, NKG2C, GITR, CD137, HVEM, SLAM, TIM1, Galectin-9, a ligand that specifically binds with CD83, and any combination thereof. In a particular embodiment the co-stimulatory signaling region comprises the intracellular domain of a co-stimulatory molecule selected from CD28 (SEQ ID NO.: 4), 4-1BB (SEQ ID NO.: 6), and combinations thereof.

The invention also provides an engineered cell comprising a B cell receptor like complex (i.e. a B cell receptor like protein), wherein the B cell receptor like complex comprises an extracellular antigen recognition domain, a trans-membrane domain, a CD79 protein or functional equivalent thereof, and a signaling region that controls T cell activation. The extracellular antigen recognition domain and the trans-membrane domain are derived from the same human or humanized B cell receptor protein and form a single human or humanized B cell receptor protein in the complex. The signaling region comprises a T cell signaling domain in combination with a co-stimulatory domain, and wherein the signaling region is fused to the CD79 protein or functional equivalent thereof. The CD79 protein as used herein consists of a CD79α protein, a CD79β protein, a CD79αβ heterodimer, or any functional equivalent thereof, including the different embodiments as mentioned hereinbefore. In another embodiment the T cell signaling domain and the co-stimulatory domain are fused to one another. In one embodiment, said cell comprises a nucleic acid sequence encoding a B cell receptor like complex according to the different embodiments of the present invention.

A co-stimulatory domain can be fully human or humanized. A co-stimulatory domain can also be a part of the complete protein. In some cases, a co-stimulatory domain can be a functional fragment of the complete protein. A co-stimulatory domain can also be non-human.

In another embodiment, the engineered cell comprising a B cell receptor like complex is a T cell. It is accordingly an object of the present invention to provide a T cell expressing a B cell receptor like complex according to the different embodiments of the present invention. T cells expressing said complex are further referred to as T-BCR cells.

Incorporation of a B cell receptor like complex in a T cell enables triggering of T cell based cytotoxicity originating from broad B cell receptor antigen activation. Using the cells of the present invention, binding of the antigen on the B cell receptor like complex will result in cytotoxic T cells through the presence of the signaling region that controls T cell activation. This signaling region comprises a T cell signaling domain in combination with a co-stimulatory domain, in the B cell receptor like complex. Since the extracellular antigen recognition domain and the trans-membrane domain are derived from a human or humanized B cell receptor protein and form a single human or humanized B cell receptor protein, extracellular fusion sites are not present in the ecto-domain, thereby avoiding unwanted and hazardous immune responses with these constructs during antibody mediated immune recognition. In addition, the chimeric part of this complex is only situated intracellular at the site where the CD79 protein and the signaling region are fused together.

Another aspect of the invention includes one or more vectors comprising a nucleic acid sequence encoding a B cell receptor like complex, wherein the B cell receptor like complex comprises an extracellular antigen recognition domain, a trans-membrane domain, a CD79 protein or functional equivalent thereof, and a signaling region that controls T cell activation. The extracellular antigen recognition domain and the trans-membrane domain are derived from the same human or humanized B cell receptor protein and form a single unit in the complex. In a particular embodiment, the extracellular antigen recognition domain and the trans-membrane domain form a single human or humanized B cell receptor protein. The signaling region comprises a T cell signaling domain in combination with a co-stimulatory domain, wherein the signaling region is fused to the CD79 protein or functional equivalent thereof. In another embodiment, said vector comprises a nucleic acid sequence encoding a B cell receptor like complex according to the different embodiments of the present invention. One or more vectors can be introduced into one cell.

The invention further provides a process for generating an engineered T cell comprising a B cell receptor like complex according to the different embodiments of the present invention. In one embodiment, said process comprises introducing one or more vectors or one or more nucleic acid sequences according to the different embodiments of the present invention into a T cell or T cell population. Said vector(s) comprise a nucleic acid sequence encoding a B cell receptor like complex, wherein the B cell receptor like complex comprises an extracellular antigen recognition domain, a trans-membrane domain, a CD79 protein or a functional equivalent thereof, and a signaling region that controls T cell activation. In another embodiment, said process comprises the introduction of said one or more vectors or said one or more nucleic acid sequences into a cell by non-viral gene delivery technology. In yet another embodiment, said process comprises the introduction of said one or more vectors or said one or more nucleic acid sequences into a cell by viral gene delivery technology.

Further, a pharmaceutical composition is disclosed comprising an engineered T cell comprising a B cell receptor like complex according to the different embodiments of the present invention.

The present invention further discloses an engineered T cell or said pharmaceutical composition according to the different embodiments of the invention for use as a medicine. In yet another embodiment, said engineered cell or said pharmaceutical composition are for use in a treatment of cancer.

The invention further provides methods to the use of engineered T cells genetically modified to stably express a desired B cell receptor like complex. Engineered T cells expressing the B cell receptor like complex according to the different embodiments of the present invention are referred to herein as T-BCR cells. In one aspect, a method is provided for stimulating a T cell-mediated immune response to a target cell population or tissue in a mammal. In one embodiment, this method comprises administration to a mammal an effective number of engineered cells genetically modified to express a B cell receptor like complex, whether or not in combination with a targeting molecule, wherein the B cell receptor like complex comprises an extracellular antigen recognition domain, a trans-membrane domain, a CD79 protein or functional equivalent thereof, and a signaling region that controls T cell activation. The extracellular antigen recognition domain and the trans-membrane domain are derived from the same human or humanized B cell receptor protein and form a single unit in the complex. Typical for this invention, the human or humanized B cell receptor protein is combined with a CD79 protein that is fused to a signaling region. The CD79 protein may consist of a CD79α protein, a CD79β protein, a CD79αβ heterodimer, or any functional equivalent thereof, including the different embodiments as mentioned hereinbefore. The signaling region comprises a T cell signaling domain in combination with a co-stimulatory domain, wherein the signaling region is fused to the CD79 protein. In a particular embodiment, the T cell signaling domain, the co-stimulatory domain or both are fused to one or both monomers of the CD79 protein as present. In addition, the extracellular antigen recognition domain of the B cell receptor is selected to recognize the target cell population or the targeting molecule, and wherein the extracellular antigen recognition domain or a targeting molecule binds to a surface antigen, thereby stimulating a T cell-mediated immune response in the mammal.

In another aspect, the invention also provides a method of providing anti-tumor immunity in a mammal. In one embodiment, the method comprises administering to a mammal an effective number of engineered cells genetically modified to express a B cell receptor like complex according to the different embodiments of the present invention, whether or not in combination with a targeting molecule. In one embodiment, the B cell receptor like complex comprises an extracellular antigen recognition domain, a trans-membrane domain, a CD79 protein or functional equivalent thereof, and a signaling region that controls T cell activation. The extracellular antigen recognition domain and the trans-membrane domain are derived from the same human or humanized B cell receptor protein and form a single unit in the complex. The signaling region comprises a T cell signaling domain in combination with a co-stimulatory domain, wherein the signaling region is fused to the CD79 protein. The CD79 protein is either a CD79α protein, a CD79β protein, a CD79α homodimer, a CD79β homodimer, a CD79αβ heterodimer, or any functional equivalent thereof, including the different embodiments as mentioned hereinbefore. In another embodiment, the co-stimulatory domain and the T cell signaling domain are fused to one another and to the CD79 protein. In a particular embodiment, the T cell signaling domain, the co-stimulatory domain or both are fused to one or both monomers of the CD79 protein as present. In addition, the extracellular antigen recognition domain of the B cell receptor is selected to recognize the target cell population or the targeting molecule, and wherein the extracellular antigen recognition domain or the targeting molecule binds to a surface antigen, thereby providing anti-tumor immunity in the mammal.

In yet another aspect, the invention also provides a method of treating a mammal having a disease, disorder or condition associated with an aberrant expression of an antigen. The method comprises administration to a mammal an effective number of engineered cells genetically modified to express a B cell receptor like complex according to the different embodiments of the present invention, whether or not in combination with a targeting molecule. In one embodiment the B cell receptor like complex comprises an extracellular antigen recognition domain, a trans-membrane domain, a CD79 protein or functional equivalents thereof, and a signaling region that controls T cell activation. The extracellular antigen recognition domain and the trans-membrane domain are derived from a human or humanized B cell receptor protein and form a single unit in the complex. The signaling region comprises a T cell signaling domain in combination with a co-stimulatory domain, wherein the signaling region is fused to the CD79. The CD79 protein consists of a CD79α protein, a CD79β protein, a CD79α homodimer, a CD79β homodimer, a CD79αβ heterodimer, or any functional equivalent thereof, including the different embodiments as mentioned hereinbefore. In another embodiment, the signaling region comprises a T cell signaling domain in combination with a co-stimulatory domain, wherein the T cell signaling domain and the co-stimulatory domain are fused to one another and to the CD79 protein. In a particular embodiment, the T cell signaling domain, the co-stimulatory domain or both are fused to one or both monomers of the CD79 protein as present. In addition, the extracellular antigen recognition domain of the B cell receptor is selected to recognize the target cell population or the targeting molecule, and wherein the extracellular antigen recognition domain or the targeting molecule binds to one or more surface antigens, thereby treating the mammal. In one embodiment of this method, the cell may be an autologous T cell.

Numbered embodiments of the present invention are as follows:

1. A B cell receptor like complex comprising:
an extracellular antigen recognition domain,
a trans-membrane domain,
a CD79 protein or a functional equivalent thereof, and
a signaling region that controls T cell activation;
   wherein the extracellular antigen recognition domain and the trans-membrane domain are derived from the same human or humanized B cell receptor protein, and wherein the signaling region comprises a T cell signaling domain in combination with a co-stimulatory domain, and wherein the signaling region is fused to the CD79 protein.

2. One or more isolated nucleic acid sequences together encoding a B cell receptor like complex according to numbered embodiment 1.

3. The B cell receptor like complex according to numbered embodiment 1, wherein the CD79 protein consists of a CD79α protein, a CD79β protein, a CD79α homodimer, a CD79β homodimer, a CD79αβ heterodimer, or any functional equivalents thereof.

4. The B cell receptor like complex according to numbered embodiment 1, wherein the T cell signaling domain, the co-stimulatory domain or both are fused to one or both monomers of the CD79 protein.

5. The B cell receptor like complex according to numbered embodiment 1, wherein the extracellular antigen recognition domain and the trans-membrane domain form a single human or humanized B cell receptor protein.

6. The B cell receptor like complex according to numbered embodiment 1, wherein the extracellular antigen recognition domain comprises at least one immunoglobulin chain.

7. The B cell receptor like complex according to numbered embodiment 6, wherein the immunoglobulin chain comprises a variable heavy chain.

8. The B cell receptor like complex according to numbered embodiments 6 or 7, wherein the immunoglobulin chain comprises a variable light chain.

9. The B cell receptor like complex according to numbered embodiments 6 to 8, wherein the extracellular antigen recognition domain comprises at least one variable heavy chain and at least one variable light chain.

10. The B cell receptor like complex according to numbered embodiments 5 to 9, wherein the variable heavy and variable light chains comprise IgA, IgG, IgM, IgD, IgE, or any combination thereof.

11. The B cell receptor like complex according to numbered embodiments 1 to 10, wherein the B cell receptor like complex is a single polypeptide.

12. The B cell receptor like complex according to numbered embodiments 1 to 10, wherein the B cell receptor like complex comprises two or more different polypeptides.

13. The B cell receptor like complex according to numbered embodiment 1, wherein the extracellular antigen recognition domain binds to a surface antigen.

14. The B cell receptor like complex according to numbered embodiment 13, wherein the extracellular antigen recognition domain binds to a universal epitope expressed on a targeting molecule.

15. The B cell receptor like complex of numbered embodiment 14 wherein the targeting molecule is a protein scaffold.

16. The B cell receptor like complex of numbered embodiments 14 or 15, wherein the targeting molecule is selected from the group consisting of scFv molecules, Darpin molecules, Nanobody molecules, Alphabody molecules, Centyrin molecules, Affibody molecules, heavy chain only antibodies or molecules from any other protein scaffold platform.

17. The B cell receptor like complex of any one of numbered embodiments 14 to 16, wherein the targeting molecule binds to a surface antigen.

18. The B cell receptor like complex of numbered embodiments 13 or 17, wherein the surface antigen is associated with a cell.

19. The B cell receptor like complex of numbered embodiment 18, wherein the cell is a solid tumor cell or a hematologic tumor cell.

20. The B cell receptor like complex of numbered embodiment 1, wherein the extracellular antigen binding domain and trans-membrane domain interact with the CD79 protein or a functional equivalent thereof.

21. The B cell receptor like complex of numbered embodiment 1, wherein the extracellular antigen binding domain and trans-membrane domain interact with the signaling region.

22. The B cell receptor like complex of numbered embodiment 1, wherein the extracellular antigen binding domain and trans-membrane domain interact with the CD79 protein or a functional equivalent thereof and with the signaling region. 23. The B cell receptor like complex of numbered embodiment 1, wherein the T cell signaling domain contains one or more ITAM motifs leading to T cell activation.

24. The B cell receptor like complex of numbered embodiment 23, wherein the T cell signaling domain is TCR zeta, FcR gamma, FcR beta, CD3 zeta, CD3 gamma, CD3 epsilon, CD5, CD22, CD66d, or any combination thereof.

25. The B cell receptor like complex of numbered embodiment 1, wherein the co-stimulatory domain comprises one or more fragments of the intracellular domain of a co-stimulatory molecule selected from CD27, CD28, 4-1BB, OX40, CD30, CD40L, ICOS, lymphocyte function-associated antigen (LFA-1), CD2, CD7, NKG2C, GITR, CD137, HVEM, TIM1, Galectin-9, a ligand that specifically binds with CD83, and any combination thereof.

26. The B cell receptor like complex of numbered embodiment 25, wherein the co-stimulatory domain comprises one or more fragments of the intracellular domain of CD28.

27. An engineered cell comprising a B cell receptor like complex according to any one of numbered embodiments 1 or 3 to 26.

28. The engineered cell of numbered embodiment 27, wherein the cell is a T cell.

29. The engineered cell of numbered embodiment 28, wherein the T cell is an effector T cell (T_{EFF}), effector-memory T cell (T_{EM}), central-memory T cell (T_{CM}), T memory stem cell (T_{SCM}), naïve T cell (T_{N}), or CD4⁺ T cell or CD8⁺ T cell.

30. The cell of numbered embodiment 28 or 29 wherein the engineered cell is a primary cell.

31. One or more vectors comprising a nucleic acid sequence encoding a B cell receptor like complex according to any of numbered embodiments 1 or 3 to 26.

32. One or more vectors of numbered embodiment 31 comprising a nucleic acid sequence according to numbered embodiment 2.

33. An engineered cell comprising one or more vectors according to numbered embodiments 31 or 32.

34. A process for generating an engineered cell according to any one of numbered embodiments 27 to 30 or 33, said process comprising transfecting a cell or cell population with one or more vectors according to numbered embodiment 31 or 32.

35. A pharmaceutical composition comprising an engineered cell according to any one of numbered embodiments 27 to 30 or 33.

36. An engineered cell according to any one of numbered embodiments 27 to 30 or 33 or a pharmaceutical composition according to numbered embodiment 35 for use as a medicine.

37. An engineered cell according to any one of numbered embodiments 27 to 30 or 33 or a pharmaceutical composition according to numbered embodiment 35 for use in a treatment of cancer.

38. A method for stimulating a T cell-mediated immune response to a target cell population or tissue in a mammal, the method comprising administering to a mammal an effective number of engineered cells according to any one of numbered embodiments 27 to 30 or 33 or an effective amount of a pharmaceutical composition according to numbered embodiment 35, thereby stimulating a T cell-mediated immune response in the mammal.

39. A method of providing an anti-tumor immunity in a mammal, the method comprising administering to a mammal an effective number of engineered cells according to any one of numbered embodiments 27 to 30 or 33 or an effective amount of a pharmaceutical composition according to numbered embodiment 35, thereby providing anti-tumor immunity in the mammal.

40. A method of treating a mammal having a disease, disorder or condition associated with an aberrant expression of an antigen, the method comprising administering to a mammal an effective number of engineered cells according to any one of numbered embodiments 27 to 30 or 33 or an effective amount of a pharmaceutical composition according to numbered embodiment, thereby treating the mammal.

Expressed alternatively, the present invention provides:

1. An engineered cell comprising:
at least one exogenous B cell receptor like complex comprising an extracellular antigen recognition domain, B cell trans-membrane domain; at least one trans-membrane signaling protein, and at least one T cell co-stimulatory domain fused to a signaling domain.

2. The engineered cell of numbered embodiment 1, wherein said extracellular antigen recognition domain comprises at least one B cell receptor (BCR) extracellular binding domain.

3. The engineered cell of numbered embodiment 2, wherein said extracellular antigen recognition domain comprises at least one immunoglobulin chain.

4. The engineered cell of numbered embodiment 3, wherein said immunoglobulin chain comprises a variable heavy chain.

5. The engineered cell of numbered embodiments 3 or 4, wherein said immunoglobulin chain comprises a variable light chain.

6. The engineered cell of any one of numbered embodiments 1 to 5, wherein said extracellular antigen recognition domain comprises at least one variable heavy chain and at least one variable light chain.

7. The engineered cell of any one of numbered embodiments 1 to 6, wherein said variable heavy and variable light chains comprise IgA, IgG, IgM, IgD, IgE, or any combination thereof.

8. The engineered cell of any one of numbered embodiments 1 to 7, wherein said B cell receptor like complex is a single polypeptide.

9. The engineered cell of any one of numbered embodiments 1 to 8, wherein said B cell receptor like complex comprises two or more different polypeptides.

10. The engineered cell of any one of numbered embodiments 1 to 9, wherein said engineering cell binds a target.

11. The engineered cell of numbered embodiment 10, wherein said target is a cell.

12. The engineered cell of numbered embodiment 11, wherein said cell has an antigen.

13. The engineered cell of numbered embodiment 12, wherein said antigen has more than one epitope.

14. The engineered cell of any one of numbered embodiments 1 to 13, wherein said engineered cell binds a cancerous cell.

15. The engineered cell of numbered embodiment 10, wherein said target is a targeting molecule.

16. The engineered cell of numbered embodiment 15, wherein the targeting molecule is a protein scaffold.

17. The engineered cell of numbered embodiment 15, wherein the targeting molecule is selected from the group consisting of scFv molecules, Darpin molecules, Nanobody molecules, Alphabody molecules, Centyrin molecules, Affibody molecules, heavy chain only antibodies, or molecules from any other protein scaffold platform.

18. The engineered cell of any one of numbered embodiments 15 to 17, wherein the targeting molecule binds to a surface antigen.

19. The engineered cell of any one of numbered embodiments 1 to 18, wherein said B cell receptor like complex is humanized.

20. The engineered cell of any one of numbered embodiments 1 to 18, wherein said B cell receptor like complex is fully human.

21. The engineered cell of any one of numbered embodiments 1 to 20, wherein said extracellular antigen recognition domain interacts with a trans-membrane signaling complex.

22. The engineered cell of numbered embodiment 21, wherein said interaction activates said trans-membrane signaling complex.

23. The engineered cell of any one of numbered embodiments 1 to 22, wherein said at least one trans-membrane signaling protein is the CD79α chain and CD79β chain complex.

24. The engineered cell of any one of numbered embodiments 1 to 23, wherein said at least one trans-membrane signaling protein is a structural equivalent or functional equivalent of said CD79α chain and CD79β chain complex.

25. The engineered cell of numbered embodiments 23 or 24, wherein said CD79α chain and CD79β are each independently fused to said at least one signaling domain.

26. The engineered cell of any one of numbered embodiments 1 to 25, wherein said signaling domain has one or more immunoreceptor tyrosine-based activation motifs (ITAMs).

27. The engineered cell of any one of numbered embodiments 1 to 26, wherein said signaling domain is TCR zeta, FcR gamma, FcR beta, CD3 gamma, CD3 delta, CD3 epsilon, CDS, CD22, CD79a, CD79b, CD66d, or any combination thereof.

28. The engineered cell of any one of numbered embodiments 1 to 27, wherein said at least one T cell co-stimulatory domain is selected from CD27, CD28, 4-1BB, OX40, CD30, CD40L, ICOS, lymphocyte function-associated antigen (LFA-1), CD2, CD7, NKG2C, GITR, CD137, HVEM, TIM1, Galectin-9, a ligand that specifically binds with CD83, and any combination thereof.

29. The engineered cell of any one of numbered embodiments 1 to 28, wherein said engineered cell is an immune cell.

30. The engineered cell of any one of numbered embodiments 1 to 29, wherein said engineered cell is a T cell.

31. The engineered cell of any one of numbered embodiments 1 to 30, wherein said engineered cell is an effector T cell (T_{EFF}), effector-memory T cell (T_{EM}), central-memory T cell (T_{CM}), T memory stem cell (T_{SCM}), naïve T cell (T_{N}), or CD4⁺ T cell or CD8⁺ T cell.

32. The engineered cell of any one of numbered embodiment 1 to 31, wherein said engineered cell is a primary cell.

33. The engineered cell of any one of numbered embodiments 1 to 32, wherein said engineered cell is formulated into a pharmaceutical composition.

34. The engineered cell of any one of numbered embodiments 1 to 33, wherein said engineered cell is formulated into a pharmaceutical composition and used to treat a subject in need thereof.

35. A method of making an engineered cell comprising:
introducing into a cell one or more polynucleic acids encoding an engineered B cell receptor like complex comprising: an extracellular antigen recognition domain, B cell trans-membrane domain, at least one trans-membrane signaling domain, at least one T cell co-stimulatory domain fused to a signaling domain.

36. The method of numbered embodiment 35, wherein said polynucleic acid encoding an extracellular antigen recognition domain, B cell trans-membrane domain, at least one trans-membrane signaling protein, at least one T cell co-stimulatory domain fused to a signaling domain are introduced into said cell with one or more vectors.

37. The method of numbered embodiments 35 or 36, wherein said polynucleic acid encoding an extracellular antigen recognition domain, B cell trans-membrane domain, at least one trans-membrane signaling protein, at least one T cell co-stimulatory domain fused to a signaling domain are introduced into said cell using non-viral techniques.

38. A pharmaceutical composition comprising said engineered cell of any one of numbered embodiments 1 to 34.

39. A method of treating a condition in a subject in need thereof comprising administering to said subject a therapeutically effective amount of said pharmaceutical composition comprising numbered embodiment 38.

40. The method of numbered embodiment 39, wherein said subject in need thereof is afflicted with cancer.

41. One or more polynucleic acids encoding at least one exogenous B cell receptor like complex comprising:

a) at least one sequence encoding for an extracellular antigen recognition domain;

b) at least one sequence encoding for a B cell trans-membrane domain;

c) at least one sequence encoding for a trans-membrane signaling protein; and

d) at least one sequence encoding for a T cell co-stimulatory domain comprising a signaling domain.

42. The one or more polynucleic acids of numbered embodiment 41, wherein said sequence encoding for an extracellular antigen recognition domain comprises at least one immunoglobulin chain sequence.

43. The one or more polynucleic acids of numbered embodiment 42, wherein said immunoglobulin chain comprises a variable heavy chain.

44. The one or more polynucleic acids of numbered embodiments 42 or 43, wherein said extracellular antigen recognition domain comprises at least one variable heavy chain and at least one variable light chain.

45. The one or more polynucleic acids of any one of numbered embodiments 42 to 44, wherein said variable heavy and variable light chains comprise IgA, IgG, IgM, IgD, IgE, or any combination thereof.

46. The one or more polynucleic acids of any one of numbered embodiments 42 to 45, wherein said B cell receptor like complex is a single polypeptide.

47. The one or more polynucleic acids of any one of numbered embodiments 42 to 46, wherein said B cell receptor like complex comprises two or more different polypeptides.

48. The one or more polynucleic acids of any one of numbered embodiments 42 to 47, wherein said B cell receptor like complex comprises a partial sequence.

49. The one or more polynucleic acids of any one of numbered embodiments 42 to 48, wherein said sequence encoding for said B cell receptor like complex is humanized.

50. The one or more polynucleic acids of any one of numbered embodiments 42 to 49, wherein said sequence encoding for said B cell receptor like complex is fully human.

51. The one or more polynucleic acids of any one of numbered embodiments 42 to 50, wherein said sequence encoding for a trans-membrane signaling protein comprises a CD79 sequence.

52. The one or more polynucleic acids of any one of numbered embodiments 42 to 51, wherein said sequence encoding for a trans-membrane signaling protein comprises a CD79 alpha chain and a CD79 beta chain.

53. The one or more polynucleic acids of any one of numbered embodiments 42 to 52, wherein said signaling domain sequence comprises one or more immunoreceptor tyrosine-based activation motif (ITAMs) sequences.

54. The one or more polynucleic acids of any one of numbered embodiments 42 to 53, wherein said signaling domain is TCR zeta, FcR gamma, FcR beta, CD3 gamma, CD3 delta, CD3 epsilon, CDS, CD22, CD79a, CD79b, CD66d, or any combination thereof.

55. The one or more polynucleic acids of any one of numbered embodiments 42 to 54, wherein said at least one T cell co-stimulatory domain is selected from CD27, CD28, 4-1BB, OX40, CD30, CD40L, ICOS, lymphocyte function-associated antigen (LFA-1), CD2, CD7, NKG2C, GITR, CD137, HVEM, TIM1, Galectin-9, a ligand that specifically binds with CD83, and any combination thereof.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1 Schematic representation of the B cell receptor like complex.

The representative B cell receptor like complex comprises an extracellular antigen recognition domain, a trans-membrane domain, a CD79 protein or functional equivalent thereof, and a signaling region that controls T cell activation. The extracellular antigen recognition domain and the trans-membrane domain are derived from the same B cell receptor and they form a single unit in the complex. The signaling region comprises a T cell signaling domain in combination with a co-stimulatory domain. The signaling region is fused to the CD79 protein.

FIG. 2 Protein sequences of the construct used to express the CD79/CD28/CD3 complex.

FIG. 3 Schematic illustration of the transgenes comprising the CD20-specific B cell receptor like complexes.

FIG. 4 Expression of a CD20-specific T-BCR in primary human T cells.

(A) Expression levels of GFP and Kathuska fluorescent molecules in primary human T cells retrovirally modified with pMP71-CD79αβ-IRES-GFP and pMP71-CD20mAb-IRES-Katushka, respectively. FACS plots depict live, CD8⁺ T cells.

(B) Expression of human IgG on the surface of primary human T cells. Histogram depicts IgG expression in the different quadrants of the FACS plot in (A).

FIG. 5 Recognition of a B cell lymphoma cells by primary human T cell modified with a CD20 specific T-BCR.

Human T cells modified with different variants of a T-BCR complex were cultured in the presence of Raji B cell line and intracellular and secreted IFN-y levels were quantified as a marker for T cell activation mediated by the T-BCR receptor.

(A) Intracellular expression of IFN-γ by T cells modified with different variants of a T-BCR complex after stimulation with Raji B cell line. FACS plots depict single, live, CD79αβ-IRES-GFP modified cells.

(B) Percentage of CD8⁺ IFN-γ⁺ T cells of the total number of live CD79αβ⁺ T cells as calculated from FACS plots from (A).

(C) IFN-γ concentration in culture supernatant after stimulation of T cells modified with different variants of a T-BCR receptor complex with Raji B cell line.

FIG. 6. Schematic illustration of different transgenes used in various T-BCR complexes.

FIG. 7. Protein sequences of the construct used to express the CD79/4-1BB/CD3 complex. Protein sequence is depicted as SEQ ID No. 9.

FIG. 8. Protein sequences of the construct used to express the CD79/4-1BB/CD28/CD3 complex. Protein sequence is depicted as SEQ ID No. 10.

FIG. 9 Protein sequences of the construct used to express CD79 wildtype complex. Protein sequence is depicted as SEQ ID NO. 11.

FIG. 10 Tumor recognition of CD20-specific T-BCR using CD28 and 4-1BB derived signaling domains in primary human T cells.

Primary T cells were retrovirally engineered with pB:CD20mAb_NEO in combination with pB:CD79_CD28CD3ζ_PURO, pB:CD79_4-1BBCD3ζ_PURO, pB:CD79CD28CD3ζ4-1BBCD3ζ_PURO or pB:CD79WT_PURO. Following introduction of transgenes T cells were cultured in the presence of geneticin and puromycin and expanded using a rapid expansion protocol (REP). After 2 weeks of expansion T cells were co-cultured with tumor cells for 24 hours at 37° C. and IFNγ secretion was measured by ELISPOT (A) or ELISA (B). Tumor cells used were K562 (Chronic Myeloid Leukemia; CD19⁻ CD20⁻), Daudi (B cell lymphoma; CD19⁺⁺, CD20⁺⁺), Raji (B cell lymphoma; CD19⁺⁺, CD20⁺⁺) and RPM18226/S (Multiple Myeloma; CD19⁻, CD20^{-/+}). A. Effector and target cells were incubated at an E:T ratio of 1:3 and IFNγ spots per 15.000 T cells is shown as mean of triplicates (+SEM). B. Effector and target cells were incubated at an E:T ratio of 1:1 in triplicates, supernatant was harvested pooled and IFNγ production was measured by ELISA.

FIG. 11 Tumor recognition of CD19-specific T-BCR in primary human T cells.

Primary T cells were retrovirally engineered with pB:CD19mAb_NEO in combination with pB:CD79_CD28CD3ζ_PURO or pB:CD79WT_PURO. Following introduction of transgenes T cells were cultured in the presence of geneticin and puromycin and expanded using a rapid expansion protocol (REP). After 2 weeks of expansion T cells were co-cultured with tumor cells for 24 hours at 37° C. and IFNγ secretion was measured by ELISPOT (A) or ELISA (B).

Tumor cells and assays as described in legends to FIG. 10.

### DETAILED DESCRIPTION OF THE INVENTION

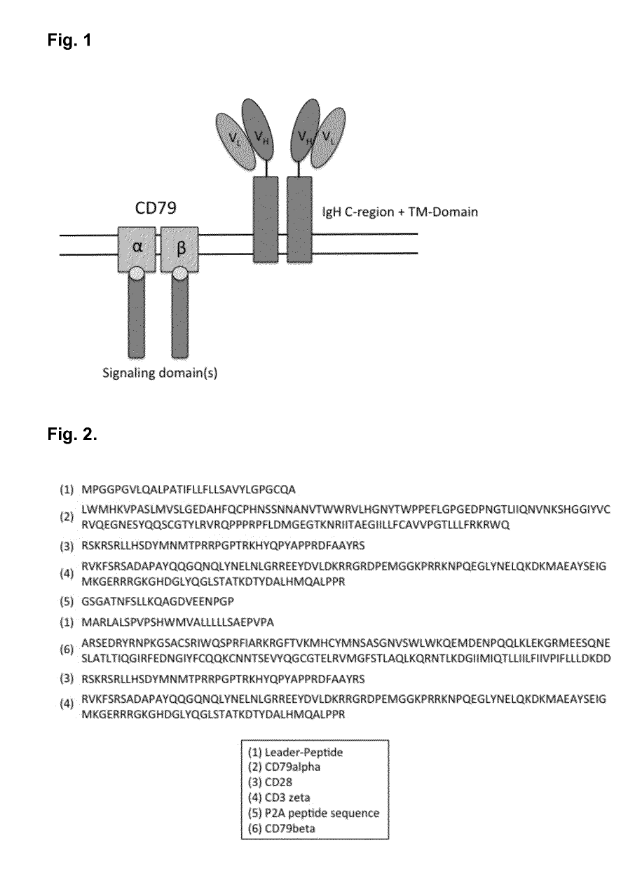

The present invention is directed to compositions and methods for immunotherapy, including but not limited to cancer, using a human or humanized B cell like receptor complex (FIG. 1). This B cell like receptor complex makes use of human or humanized B cell receptor constructs. Typical for this invention, the human or humanized B cell receptor is combined with a CD79 protein or functional equivalent thereof, and a signaling region that controls T cell activation.

### DEFINITIONS

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. In certain cases, a T cell can require at least two signals to become fully activated. The first signal can occur after engagement of a TCR by the antigen-MHC complex, and the second signal can occur by engagement of co-stimulatory molecules. In some cases, Anti-CD3 can mimic the first signal and anti-CD28 can mimic the second signal in vitro. For example, an engineered T cell can be activated by an expressed BCR.

The term “adjacent” and its grammatical equivalents as used herein can refer to right next to the object of reference. For example, the term adjacent in the context of a nucleotide sequence can mean without any nucleotides in between. For instance, polynucleotide A adjacent to polynucleotide B can mean AB without any nucleotides in between A and B.

The term “antigen” or “Ag”, and their grammatical equivalents as used herein, can refer to a molecule that provokes the immune response. This immune response may involve either antibody production, or the activation of specific immunologically-competent cells, or both. The skilled artisan will understand that any macromolecule or macromolecular complex, including virtually all proteins or peptides, can serve as an antigen. For example, a tumor cell antigen can be recognized by a BCR.

The term “immunoglobulin” or “Ig”, and their grammatical equivalents as used herein can refer to a class of proteins, which function as antibodies. Antibodies expressed by B cells are sometimes referred to as the B cell receptor or antigen receptor. The five members included in this class of proteins are IgA, IgG, IgM, IgD, and IgE, of which IgG is the most common circulating antibody. It is the most efficient immunoglobulin in agglutination, complement fixation, and other antibody responses, and is important in defense against bacteria and viruses. An immunoglobulin can be part of any class or a chimera of different classes. An immunoglobulin for example can contain portions of IgA and IgG. An immunoglobulin can be fully human, humanized, or non-human.

The term “autologous” and its grammatical equivalents as used herein can refer to as originating from the same being. For example, a sample (e.g. cells) can be removed, processed, and given back to the same subject (e.g. patient) at a later time. An autologous process is distinguished from an allogeneic process where the donor and the recipient are different subjects.

The term “B cell receptor” as used herein, refers to an immunoglobulin molecule or antibody that specifically binds with an antigen and is attached to the surface of a B cell. Antibodies can occur in two physical forms, a soluble form that is secreted from the cell, and a membrane-bound form that is attached to the surface of a B cell and is referred to as the B cell receptor. The B cell receptor can be found on the surface of B cells and facilitates activation of B cells and their subsequent differentiation into either plasma cells, or memory B cells. Antibodies can also be intact immunoglobulins derived from natural sources or from recombinant sources and can be immunoreactive portions of intact immunoglobulins. A B cell receptor can be human or non-human.

The term “epitope” and its grammatical equivalents as used herein can refer to a part of an antigen that can be recognized by antibodies, B cell, T cells or engineered cells. For example, an epitope can be a cancer epitope that is recognized by a BCR. Multiple epitopes within an antigen can also be recognized. The epitope can also be mutated.

The term “engineered” and its grammatical equivalants as used herein can refer to one or more alterations of a nucleic acid, e.g. the nucleic acid within an organism's genome. The term “engineered” can refer to alterations, additions, and/or deletions of genes. An engineered cell can also refer to a cell with an added, deleted and/or altered gene.

The term “function” and its grammatical equivalents as used herein can refer to the capability of operating, having, or serving an intended purpose. The term functional can comprise any percent from baseline to 100% of normal function. For example, functional can comprise having 5%, 25%, 50%, 75% and/or up to 100% of normal function.

The term “functional equivalent” and its grammatical equivalents as used herein can refer to proteins or fragments of protein that perform its intended purpose (below, at, or above its normal function). For example, the CD3 protein or the CD79 protein can exhibit trafficking and/or signaling activity that is substantially equivalent to either the CD3 protein or the CD79 protein from which they are derived, including proteins having a substantially identical sequence to either of the CD79 protein, the CD3 protein or fragments of said proteins.

The term “fused” and its grammatical equivalents as used herein can refer to the joining of two proteins or fragments. For example, “fused” can refer to the joining of two entities such that they are adjacent to each other after being fused. “Fused” can also refer to the joining of two entities such that they are not in contact with each other but separated, for example, 1 to 1000 bases (in polynucleotides) or 1 to 350 amino acids (in a polypeptide).

The term “humanized B cell receptor” and its grammatical equivalents as used herein can refer to a B cell receptor or antibody derived from a non-human species whose protein sequences have been modified to increase their similarity to antibody variants produced naturally in humans. For example, a humanized B cell receptor can comprise all of at least one, and typically two, variable domains (Fab, Fab′, F(ab′)₂, Fabc, Fv) in which all or substantially all of the CDR regions correspond to those of a non-human immunoglobulin and all or substantially all of the Fc regions are those of a human immunoglobulin consensus sequence. The humanized immunoglobulin molecules of the present invention can be isolated from a transgenic non-human animal engineered to produce humanized immunoglobulin molecules. Humanized immunoglobulins or antibodies can include immunoglobulins (Igs) and antibodies that are further diversified through gene conversion and somatic hypermutations in gene converting animals.

The terms “nucleic acid”, “polynucleotide”, “polynucleic acid”, and “oligonucleotide” and their grammatical equivalents can sometimes be used interchangeably and can refer to a deoxyribonucleotide or ribonucleotide polymer, in linear or circular conformation, and in either single- or double-stranded form. For the purposes of the present disclosure, these terms should not to be construed as limiting with respect to length. The terms can also encompass analogues of natural nucleotides, as well as nucleotides that are modified in the base, sugar and/or phosphate moieties (e.g., phosphorothioate backbones). In general, an analogue of a particular nucleotide can have the same base-pairing specificity, i.e., an analogue of A can base-pair with T. The terms can also refer to fragments of mature proteins and modifications or derivatives thereof, such as glycosylated versions of such polynucleic acids, polynucleic acids encoding a signal peptide, truncated polynucleic acids having comparable biological activity and the like.

The term “phenotype” and its grammatical equivalents as used herein can refer to a composite of an organism's observable characteristics or traits, such as its morphology, development, biochemical or physiological properties, phenology, behavior, and products of behavior. Depending on the context, the term “phenotype” can sometimes refer to a composite of a population's observable characteristics or traits.

The term “recipient” and their grammatical equivalents as used herein can refer to a human or non-human animal. The recipient can also be in need thereof.

The term “substantially identical” and its grammatical equivalents as used herein can refer to a sequence that is an amino acid or nucleotide sequence that differs from a reference sequence by one or more conservative substitutions or by one or more non-conservative substitutions, deletions, or insertions located at positions of the sequence that do not destroy the biological function of the amino acid or nucleic acid molecule. Such a sequence can at least 50%, 55%, 60%, 65%, 75%, 80%, 85%, 90%, or 95%, or as much as 96%, 97%, 98%, or 99% identical when optimally aligned at the amino acid or nucleotide level to the sequence used for comparison using for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. The length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and Pretty Box. Such software matches similar sequences by assigning degrees of homology to various substitutions, deletions, substitutions, and other modifications. Alternatively, or additionally, two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO4, pH 7.2, 7% SDS, 1 mM EDTA, and 1% BSA (fraction V), at a temperature of 65[deg.]C., or a buffer containing 48% formamide, 4.8×SSC, 0.2 M Tris-Cl, pH 7.6, 1×Denhardt's solution, 10% dextran sulfate, and 0.1% SDS, at a temperature of 42[deg.]C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N. Y., 1998, which is hereby incorporated by reference. The length of comparison sequences can also be at least 60, 70, 80, or 90 nucleotides, or over 100, 200, or 500 nucleotides. The substantially identical sequence can refer to a human or non-human sequence.

The term “T cell activation” or “T cell triggering” and its grammatical equivalents as used herein, can refer to the state of a T cell that has been sufficiently stimulated to induce detectable cellular proliferation, cytokine production and/or detectable effector function, such as phosphorylation of signaling pathway proteins. In the context of the current invention, “full T cell activation” is similar to triggering T cell cytotoxicity. T cell activation can be measured using various assays known in the art. Said assays can be an ELISA to measure cytokine secretion, an ELISPOT, flow cytometry assays to measure intracellular cytokine expression (CD107), flow cytometry assays to measure proliferation, and cytotoxicity assay (51Cr release assay) to determine target cell elimination. Said assays typically use controls (non-engineered cells) to compare to engineered cells (T-BCR) to determine relative activation of an engineered cell compared to a control. Additionally, said assay can compare engineered cells incubated or put into contact with a target cell not expressing the target antigen. For example, the comparison can be a CD19 T-BCR cell incubated with a target cell that does not express CD19.

The term “transgene” and its grammatical equivalents as used herein can refer to a gene or genetic material that is transferred into an organism. For example, a transgene can be a stretch or segment of DNA containing a gene that is introduced into an organism. When a transgene is transferred into an organism, the organism can be then referred to as a transgenic organism. A transgene can retain its ability to produce RNA or polypeptides (e.g., proteins) in a transgenic organism. A transgene can be composed of different nucleic acids, for example RNA or DNA. A transgene may encode for an engineered B cell receptor like complex, for example a BCR transgene. A transgene may comprise a signaling domain.

### OVERVIEW

Disclosed herein are compositions and methods useful for genetically modifying cells and nucleic acids for therapeutic applications. The compositions and methods described throughout can use a nucleic acid-mediated genetic engineering process for tumor-specific BCR expression. Effective adoptive cell transfer-based immunotherapies (ACT) can be useful to treat cancer (e.g., metastatic cancer) patients. For example, autologous peripheral blood lymphocytes (PBL) can be modified using non-viral or viral methods to express a B cell receptor (BCR) that recognizes unique antigens on cancer cells and can be used in the disclosed compositions and methods. The present invention is directed to compositions and methods for immunotherapy, including but not limited to cancer, using a human or humanized

B cell like receptor complex (FIG. 1). This B cell like receptor complex makes use of human or humanized B cell receptor constructs. Typical for this invention, the human or humanized B cell receptor is combined with a CD79 protein or functional equivalent thereof, and a signaling region that controls T cell activation.

The B cell receptor like complex of the present invention can be comprised of an extracellular antigen recognition domain and a trans-membrane domain derived from a human or humanized B cell receptor, and a CD79 protein or functional equivalent thereof, and a signaling region that controls T cell activation. The signaling region comprises a T cell signaling domain in combination with a co-stimulatory domain. Typically, the signaling region is fused to the CD79 protein. Furthermore, in some embodiments of the invention, the B cell like receptor complex of the present invention utilizes a targeting molecule as the bridge between cytotoxic T cells and targeted cells.

The present invention differs from the traditional CARs in two important features: (1) the extracellular antigen recognition domain and trans-membrane domain are derived from the same human or humanized B cell receptor and they form a single B cell receptor, and (2) the signaling region is fused to the CD79 protein.

The extracellular domain or ecto-domain of a typical CAR consists of the single-chain variable fragment (scFv) from the antigen binding sites of a monoclonal antibody, thereby linking the V_{H} and V_{L} domains. The scFv is linked to a flexible trans-membrane domain followed by one or more endo-domains that may include a tyrosine-based activation motif such as that from CD3 zeta. In the so-called second and third generation CARs, additional activation domains from co-stimulatory molecules such as CD28 and CD137 (4-1BB), which serve to enhance T cell survival and proliferation, were included. Because of the fusion of protein domains derived from different proteins, unwanted immune response that can jeopardize the therapeutic effects can occur when using these CAR constructs. In contrast, in the present invention, the extracellular antigen recognition domain and the trans-membrane domain are derived from the same human or humanized B cell receptor protein and additionally form a single unit in the complex. As a result, no fusion sites are present in the ecto-domain of these constructs, thereby avoiding unwanted and hazardous immune responses. Furthermore, the signaling region, comprising one or more ITAM motifs in combination with co-stimulatory molecules leading to T cell activation, is not linked to the B cell receptor but it is fused to the CD79 protein.

The B cell receptor like complex of the present invention is comprised of an extracellular antigen recognition domain, a trans-membrane domain, a CD79 protein or functional equivalent thereof, and a signaling region that controls T cell activation. In one embodiment, the extracellular antigen recognition domain and trans-membrane domain can be fully human. In other cases, the extracellular antigen recognition domain and trans-membrane domain can be humanized. In other cases, the extracellular antigen recognition domain and trans-membrane can be non-human. Typical for the present invention, the extracellular antigen recognition domain and trans-membrane domain are derived from the same human or humanized B cell receptor protein and form a single unit in the complex.

In the present invention, the B cell receptor like complex comprises an extracellular antigen recognition domain and a trans-membrane domain that are derived from the same human or humanized B cell receptor. In one embodiment, the extracellular antigen recognition domain and trans-membrane domain form a fully human or humanized B cell receptor or immunoglobulin. As said, typical for this invention, the full immunoglobulin or B cell receptor forms a single unit in the complex. This is an important difference as compared to the current CAR constructs. In the currently available CARs, the extracellular antigen recognition domain, often not fully human, is fused to a trans-membrane domain of a different protein. This can introduce unwanted immune responses due to the formation of neo-epitopes, which can jeopardize the therapeutic effects. In contrast, in the present invention, the B cell receptor like complex comprises an extracellular antigen recognition domain and a trans-membrane domain that forms one single human or humanized protein, thereby avoiding toxic and allergic reactions.

In some cases, a B cell receptor like complex can contain a cell-surface immunoglobulin. A cell-surface immunoglobulin can be the binding region of said B cell receptor like complex. A binding region can utilize heavy and light chains. A heavy and light chain can be derived from IgA, IgG, IgM, IgD, IgE, or any combination thereof. A heavy and light chain can be derived from partial fragments of IgA, IgG, IgM, IgD, IgE, or any combination thereof. In some cases, subclasses of IgA, IgG, IgM, IgD, or IgE can be used.

In some cases, the variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

In some cases, a class of an immunoglobulin can refer to the type of constant domain or constant region possessed by its heavy chain. There are five major classes of immunoglobulins: IgA, IgD, IgE, IgG, and IgM, and several of these may be further divided into subclasses (isotypes), e.g., IgG, IgG₂, IgG₃, IgG₄, IgA₁, and IgA₂. The heavy chain constant domains that correspond to the different classes of immunoglobulins can be called α, δ, ε, γ, and μ, respectively. An immunoglobulin of the present invention can be of any class or subclass described herein. An immunoglobulin of the present invention can be a partial fragment of any class or subclass described herein. An immunoglobulin of the present invention can be a chimera of an immunoglobulin class or subclass described herein.

In some cases, variants of said cell surface immunoglobulin are included herein. A variant can refer to mean nucleic acid sequences that allow for the degeneracy of the genetic code, nucleic acid sequences that can encode for a polypeptide sequence that can comprise amino acid substitutions of functionally equivalent residues and/or mutations that enhance the functionality of the extracellular immunoglobulin domain. In some cases, the said functionality of the extracellular domain can include, but is not limited to, formation of a BCR capable of signal transduction. By allowing for the degeneracy of the genetic code, the invention encompasses sequences that have at least 50%, or more sequence identity to the extracellular polypeptide sequence of an immunoglobulin.

In one embodiment of the invention, the B cell receptor like complex binds directly to a surface antigen present on target cells or tissue. In a particular embodiment, the surface antigen can be a tumor antigen, also called tumor-associated antigen. In particular, the B cell receptor like complex binds directly to an epitope of an antigen. More in particular, said epitope can be a tumor cell epitope. Such a tumor cell epitope may be derived from a wide variety of tumor antigens such as antigens from tumors resulting from mutations, shared tumor specific antigens, differentiation antigens, and antigens overexpressed in tumors. Tumor-associated antigens may be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens may be, for example, proteins or protein fragments, complex carbohydrates, gangliosides, haptens, nucleic acids, other biological molecules or any combinations thereof. A tumor antigen is an antigen produced in tumor cells thereby triggering an immune response triggered in the host. Neo-antigens are also considered as tumor antigens. Neo-antigens are a class of tumor antigens, which arise from tumor-specific mutations in an expressed protein. Known tumor antigens include but are not limited to, CD19, CD20, CD22, HER-1, HER-2, HER-3, ROR-1, mesothelin, CD33/IL-3Ra, c-Met, PSMA, PSCA, gp100, WT1, CD22, CD171, Glycolipid F77, EGFRvIll, GD-2, NY-ESO-1 TCR, MAGE-A3 TCR. In the present invention, the tumor antigen is selected from the group of available tumor antigens, and any combination thereof.

In another embodiment, the B cell receptor like complex utilizes a targeting molecule as the bridge between cytotoxic T-BCR cells and targeted cells. The targeting molecule is a molecule that is recognized by the extracellular antigen recognition domain of the B cell receptor like complex. Hence in said instance, the B cell receptor like complex binds to a universal epitope present on the targeting molecule. The targeting molecule itself recognizes an antigen present on the target cell or tissue. Exemplary tumor-targeting molecules are scFv molecules, Darpin molecules, Nanobody molecules, Alpha body molecules, Centyrin molecules, Affibody molecules, heavy chain only antibodies or molecules from any other scaffold platform. scFv molecules are single chain variable fragments. They are fusion proteins of the variable regions of the heavy and light chains of immunoglobulins, connected with a short linker peptide of 10-25 amino acids. DARPins are genetically engineered antibody mimetic proteins typically exhibiting highly specific and high-affinity target protein binding. They are derived from natural ankyrin proteins and consist of at least 3, usually 4 or 5, repeat motifs of these proteins. Nanobodies or single-domain antibodies are antibody fragments consisting of a single monomeric variable antibody domain. They are able to bind selectively to a specific antigen. Centyrins are small, simple, highly stable single domain proteins with a structural homology to antibody variable domains. Affibody ® molecules are a novel class of antibody mimetics with superior characteristics surpassing mAbs and antibody fragments. Heavy chain only antibodies are antibodies that consist only of two heavy chains (V_{H}) and lack the two light chains (V_{L}). These heavy chain only antibodies can still bind antigens despite having only V_{H} domains.

The signaling region of said B cell receptor like complex is responsible for activation of at least one of the normal effector functions of the T cell in which the B cell receptor like complex has been placed in. In the present invention, the signaling region of the B cell receptor like complex comprises a T cell signaling domain in combination with a co-stimulatory domain. The signaling region is fused to the CD79 protein or functional equivalent thereof. The CD79 protein consists of a CD79α protein, a CD79β protein, CD79α homodimer, a CD79β homodimer, a CD79αβ heterodimer, or any functional equivalent thereof. In one embodiment of the present invention, the signaling region is fused to one or both monomers of the CD79 protein or functional equivalent thereof. In another embodiment the T cell signaling domain and the co-stimulatory domain are fused to one another thereby composing the signaling region. In an even further embodiment said fused T cell signaling domain, the co-stimulatory domain or both are further fused to one or both monomers of the CD79 protein.

As said, typical for this invention and different from what is known from the prior-art, the B cell receptor forms a complex with a CD79 protein that is fused to a signaling region. CD79 is a trans-membrane protein that functions as the signaling component of the B cell receptor (BCR). The BCR is a multimeric complex that includes the antigen-specific component referred to as a surface immunoglobulin (slg). The slg associates non-covalently with two other proteins, CD79α (Ig-α) and CD79β (Ig-β), which are necessary for expression and function of the BCR complex. CD79α and CD79β, as a heterodimer stabilized by disulphide binding, comprise a key component of the BCR involved in regulating B cell development and activity in vivo. Upon B cell receptor binding, CD79α and CD79β become phosphorylated on tyrosine residues of the ITAM region, as well as on serine and threonine residues on CD79α. CD79β enhances phosphorylation of CD79α, possibly by recruiting kinases that phosphorylate CD79α or by recruiting proteins that bind to CD79α and protects it from dephosphorylation. Active CD79α, in turn, stimulates downstream signaling pathways involved in BCR signaling. As used herein, the CD79 trans-membrane protein is primarily directed to human CD79 and its isoforms, also known as the human B-cell antigen receptor complex-associated protein, wherein the amino acid sequence of the human CD79α and its isoforms is known from SwissProt entry P11912; and wherein the amino acid sequence of the human CD79β and its isoforms is known from SwissProt entry P40259. It will be apparent to the skilled artisan that CD79 as used herein is not limited to the human CD79 and its isoforms as disclosed in the aforementioned SwissProt entries, but meant to include any functional equivalents thereof. The term ‘CD79 or functional equivalent thereof’ means all variants that are referenced above and isoforms thereof that retain their function as the signaling component of the B cell receptor as described in Campbell et al., Proc Natl Acad Sci USA, 1991, 88(9); Vasile et al., Mol Immunol 1994, 31(6)). A functional equivalent to CD79 can be a fragment, a portion, or a larger protein comprising CD79. The CD79 protein subunits can be used within the context of the present invention to substitute for the entire CD79 protein. The CD79 subunits, the CD79α and CD79β, can form a heterodimer stabilized by disulphide binding. In some cases, a functional equivalent of CD79 can replace either or both of the CD79α and CD79β, and form the heterodimer stabilized by disulphide binding. In some cases, the individual components of the CD79, the CD79α and CD79β, can be independently complexed to a functional equivalent. For example, a functional equivalent can comprise a CD79α complexed to the functional equivalent of CD79β. For example, a functional equivalent can comprise a CD79β complexed to the functional equivalent of CD79α.

As described above, further to CD79 protein, the B cell receptor like complex in the different embodiments of the present invention comprises an extracellular antigen recognition domain and a trans-membrane domain, comprising the B cell receptor or immunoglobulin, and a signaling region that controls T cell activation. The signaling region comprises a T cell signaling domain in combination with a co-stimulatory domain. As said, typical for this invention, the signaling region is fused to the CD79 protein.

One of the characteristics of the present B cell receptor like complex is the fusion of a signaling region with the CD79 protein or functional equivalent thereof. Typical, the signaling region comprises a T cell signaling domain in combination with a co-stimulatory domain. As a result, and different from the current prior-art, signaling through the B cell receptor like complex after antigen recognition results in full activation of the T cells leading to tumor cytotoxicity.

In the present invention, the T cell signaling domain may contain signaling motifs, which are known as immunoreceptor tyrosine-based activation motifs (ITAMs). Examples of ITAM containing cytoplasmic signaling sequences include those derived from TCR zeta, FcR gamma, FcR beta, CD3 gamma, CD3 zeta, CD3 delta, CD3 epsilon, CDS, CD22, CD79α, CD79β, and CD66d. In some embodiments, the T cell signaling domain is selected from the group of molecules consisting of CD3 zeta, CD3 epsilon, CD3 delta, CD3 gamma, and other CD3 like sequences, including functional equivalents thereof.

An example of a T cell signaling domain containing one or more ITAM motifs is the CD3 zeta domain (SEQ ID NO.: 3), also known as T-cell receptor T3 zeta chain or CD247. This domain is part of the T-cell receptor-CD3 complex and plays an important role in coupling antigen recognition to several intracellular signal-transduction pathways with primary effector activation of the T cell. As used herein, CD3 zeta is primarily directed to human CD3 zeta and its isoforms as known from Swissprot entry P20963, including proteins having a substantially identical sequence. As part of the B cell receptor like complex, again the full T cell receptor T3 zeta chain is not required and any derivatives thereof comprising the signaling domain of T-cell receptor T3 zeta chain are suitable in the methods of the present invention, including any functional equivalents thereof.

With respect to the co-stimulatory signaling domain in the signaling region of the B cell receptor like complex, the B cell receptor like complex can be designed to comprise several possible co-stimulatory signaling domains. As is well known in the art, in naïve T-cells the mere engagement of the T-cell receptor is not sufficient to induce full activation of T-cells into cytotoxic T-cells. Full, productive T cell activation requires a second co-stimulatory signal. Several receptors that have been reported to provide co-stimulation for T-cell activation, include, but are not limited to CD28, OX40, CD27, CD2, CDS, ICAM-1, LFA-1 (CD11a/CD18), and 4-1BB. The signaling pathways utilized by these co-stimulatory molecules share the common property of acting in synergy with the primary T cell receptor activation signal. In the B-cell receptor like complex of the present invention, the antigen presenting cells may lack the counter-receptor molecules necessary for co-stimulation. Consequently and instead of the complete co-stimulatory receptors, the B cell receptor like complex comprises the co-stimulatory signaling regions of said receptors; in particular the intracellular domain of said co-stimulatory signaling regions. Possible examples of co-stimulatory molecules suitable in the methods of the present invention include the intracellular domains of a co-stimulatory molecule selected from the group consisting of CD27, CD28, 4-1BB, OX40, CD30, CD40L, lymphocyte function-associated antigen-1 (LFA-1), CD2, CD7, NKG2C, GITR, CD137, HVEM, TIM1, Galectin-9, a ligand that specifically binds with CD83, and any combination thereof. These co-stimulatory signaling regions provide a signal that is synergistic with the primary effector activation signal, in the present invention originating from one or more ITAM motifs, for example a CD3 zeta signaling domain (SEQ ID NO.: 3), and can complete the requirements for activation of the T cell. A co-stimulatory domain can be fully human or humanized. A co-stimulatory domain can also be a part of the full protein. In some cases, a co-stimulatory domain can be a functional fragment of the full protein. A co-stimulatory domain can also be non-human.

Typical for this invention, the addition of co-stimulatory domains to the B cell receptor like complex can enhance the efficacy and durability of the engineered T-BCR cells.

In a non-modified T cell, at least two signals are necessary for full T cell activation. Signal 1 is derived from antigen recognition and binding in the context of MHC and signal 2 is coming from the simultaneous engagement of co-stimulatory molecules. T cell activation may result in T cell proliferation, cytokine production, survival, and cytotoxicity. In the intracellular part of the current B cell receptor like complex, co-stimulatory sequences are put in series with T cell signaling sequences. Upon antigen recognition by the extracellular domain of the B cell receptor, which is independent of MHC, both the activation signal and co-stimulatory signals are delivered to the T cell resulting in full T cell activation. Within the B cell receptor like complex of the present invention, simultaneous engagement of the T cell signaling domain and of the co-stimulatory domain by engagement of the extracellular antigen recognition domain of the B cell receptor (immunoglobulin) with the antigen results in full T cell activation. “T cell activation” or “T cell triggering”, as used herein, refers to the state of a T cell that has been sufficiently stimulated to induce detectable cellular proliferation, cytokine production and/or detectable effector function. In the context of the current invention, “full T cell activation” is similar to triggering T cell cytotoxicity.

Prior to or after genetic modification of the T cells to express a desirable B cell receptor like complex, the T-BCR cells can be activated and expanded generally using methods as described, for example, in U.S. Pat. Nos. 6,352,694, 6,534,055. Generally, the T cells of the invention are expanded by contact with a surface having attached thereto an agent that stimulates one or more ITAM motifs (e.g. CD3zeta) and a ligand that stimulates a co-stimulatory molecule. In particular, T-BCR cell populations may be stimulated such as by contact with an anti-CD3 antibody, or antigen-binding fragment thereof. For co-stimulation of an accessory molecule on the surface of the T-BCR cells, a ligand that binds the accessory molecule is used. For example, a population of T-BCR cells can be contacted with an anti-CD3 antibody and an anti-CD28 antibody, under conditions appropriate for stimulating proliferation of the T cells. Further, T cell activation can experimentally be induced by contact of T-BCR cells (effector cells) with target cells that will activate the T cells. Target cells are generally tumor cells naturally expressing the target to which the T-BCR complex that is expressed in the T cells is directed (e.g. CD19 or CD20). These target cells are selected from the group comprising, but not limited to, the Raji B cell lymphoma cell line, the Daudi B lymphoblast cell line or the K562 myelogenous leukemia cell line. Negative control cells are generally also used. T cell activation by effector cells can functionally be monitored with a ⁵¹Chromium-release assay (cell-mediated cytotoxicity), a proliferation assay, a cytotoxicity assay, and quantification of intracellular or secreted cytokines produced by the activated T cells (e.g. IFN-γ ELISPOT). Other methods, well-known to the person skilled in the art, can be used to evaluate T cell activation as well.

The invention also provides an engineered cell comprising a B cell receptor like complex (i.e. a B cell receptor like protein), according to the different embodiments of the present invention. In another embodiment, the engineered cell comprising a B cell receptor like complex is a T cell. In addition, the present invention relates generally to the use of engineered T cells genetically modified to stably express a desired B cell receptor like complex. It is accordingly an object of the present invention to provide an engineered T cell expressing a B cell receptor like complex according to the different embodiments of the present invention. T cells expressing the B cell receptor like complex according the different embodiments of the present invention are referred to herein as T-BCR cells. In one embodiment, viral or non-viral gene delivery methods known to the skilled person in the field are used for generation of T-BCR cells. One or more vectors can be introduced into one T cell. The T-BCR cells of the invention are able to replicate in vivo resulting in long-term persistence that can lead to sustained tumor control.

The invention further provides a process for generating an engineered T cell comprising a B cell receptor like complex according to the different embodiments of the present invention. Said process comprises introducing one or more vectors or one or more nucleic acid sequences according to the different embodiments of the present invention into a T cell or T cell population. Said vectors comprise a nucleic acid sequence encoding a B cell receptor like complex, wherein the B cell receptor like complex comprises an extracellular antigen recognition domain, a trans-membrane domain, a CD79 protein or a functional equivalent thereof, and a signaling region that controls T cell activation. In another embodiment, said process comprises the introduction of said one or more vectors or said one or more nucleic acid sequences into a cell by non-viral gene delivery technology. In yet another embodiment, said process comprises the introduction of said one or more vectors or said one or more nucleic acid sequences into a cell by viral gene delivery technology. The processes of viral or non-viral gene delivery may be done by any convenient manner known by the person skilled in the art.

A number of viral based systems have been developed for gene transfer into mammalian cells. For example, retroviruses provide a convenient platform for gene delivery systems. A selected gene can be inserted into a vector and packaged in retroviral particles using techniques known in the art. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells. They also have the added advantage of low immunogenicity.

Prior to expansion and genetic modification of the T cells of the invention, a source of T cells is obtained from a subject. T cells can be obtained from a number of sources, including PBMCs, bone marrow, lymph node tissue, cord blood, thymus tissue, tissue from a site of infection, ascites, pleural effusion, spleen tissue, and tumors. In certain embodiments of the present invention, T cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In a particular embodiment, the engineered cell can be a T cell. The engineered cell can be an effector (T_{EFF}), effector-memory (T_{EM}), central-memory (T_{CM}), T memory stem (T_{SCM}), naive (T_{N}), or CD4+ or CD8+. The T cells can also be selected from a bulk population, for example, selecting T cells from whole blood. The T cells can also be expanded from a bulk population. The T cells can also be skewed towards particular populations and phenotypes. The engineered cell can also be expanded ex vivo. The engineered cell can be formulated into a pharmaceutical composition. The engineered cell can be formulated into a pharmaceutical composition and used to treat a subject in need thereof. The engineered cell can be autologous to a subject in need thereof. The engineered cell can be allogenic to a subject in need thereof. The engineered cell can also be a good manufacturing practices (GMP) compatible reagent. The engineered cell can be a part of a combination therapy to treat a subject in need thereof. The engineered cell can be a human cell. The subject that is being treated can be a human.

A method of attaining suitable cells can comprise sorting cells. In some cases, a cell can comprise a marker that can be selected for the cell. For example, such marker can comprise GFP, a resistance gene, a cell surface marker, an endogenous tag. Cells can be selected using any endogenous marker. Suitable cells can be selected or sorted using any technology. Such technology can comprise flow cytometry and/or magnetic columns. The selected cells can then be infused into a subject. The selected cells can also be expanded to large numbers. The selected cells can be expanded prior to infusion.

Vectors can be delivered to cells ex vivo, such as cells explanted from an individual patient (e.g., lymphocytes, T cells, bone marrow aspirates, tissue biopsy), followed by re-implantation of the cells into a patient, usually after selection for cells which have incorporated the vector.

Prior to or after selection, the cells can be expanded.

Ex vivo cell transfection can also be used for diagnostics, research, or for gene therapy (e.g. via re-infusion of the transfected cells into the host organism). In some cases, cells are isolated from the subject organism, transfected with a nucleic acid (e.g., gene or DNA), and re-infused back into the subject organism (e.g. patient).

Further, the present embodiment also provides a pharmaceutical composition comprising one or more engineered cells comprising a B cell receptor like complex according to the different embodiments of the present invention. In one embodiment, the engineered cells or the pharmaceutical composition comprising said engineered cells are used as a medicine. In another embodiment, said engineered cells or said pharmaceutical composition are used in treatment of a cancer.

Described herein is a method of treating a disease (e.g. cancer) in a recipient comprising transplanting to the recipient one or more cells comprising engineered cells. The method disclosed herein can be used for treating or preventing disease including, but not limited to, cancer, cardiovascular diseases, lung diseases, liver diseases, skin diseases, or neurological diseases.

In one embodiment, the invention relates to administering an engineered T cell expressing a B cell receptor like complex for the treatment of a patient having cancer or at risk of having cancer using lymphocyte infusion. Preferably, autologous lymphocyte infusion is used in the treatment. Autologous peripheral blood monocytes (PBMCs) are collected from a patient in need of treatment and T cells are activated and expanded using the methods described herein and known in the art and then infused back into the patient. Populations of T-BCR cells may be formulated for administration to a subject using techniques known to the skilled artisan. Alternatively, allogeneic lymphocyte infusion can be used.

In some cases, populations of engineered T cells may be formulated for administration to a subject using techniques known to the skilled artisan. Formulations comprising populations of T-BCR cells may include pharmaceutically acceptable excipient(s). Excipients included in the formulations will have different purposes depending, for example, on the subpopulation of T cells used and the mode of administration. Examples of generally used excipients included, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents. The formulations comprising populations of T-BCR cells will typically have been prepared and cultured in the absence of any non-human components, such as animal serum.

A formulation may include one population of T-BCR cells, or more than one, such as two, three, four, five, six or more population of T-BCR cells. The formulations comprising population(s) of T-BCR cells may be administered to a subject using modes and techniques known to the skilled artisan. Exemplary modes include, but are not limited to, intravenous injection. Other modes include, without limitation, intratumoral, intradermal, subcutaneous (s.c., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection of infusion of the formulations can be used to effect such administration. The formulations comprising population(s) of T-BCR cells that are administered to a subject comprise a number of T-BCR cells that is effective for the treatment and/or prophylaxis of the specific indication or disease. Thus, therapeutically-effective populations of T-BCR cells are administered to subjects when the methods of the present invention are practiced. In general, formulations are administered that comprise between about 1×10⁴ and about 1×10¹⁰ T-BCR cells. In most cases, the formulation will comprise between about 1×10⁵ and about 1×10⁹ T-BCR cells, from about 5×10⁵ to about 5×10⁸ T-BCR cells, or from about 1×10⁶ to about 1×10⁷ T-BCR cells. However, the number of T-BCR cells administered to a subject will vary between wide limits, depending upon the location, source, identity, extent and severity of the cancer, the age and condition of the individual to be treated etc. A physician will ultimately determine appropriate dosages to be used.

Tumor-targeting molecules are administered to a subject prior to, or concurrent with, or after administration of the T-BCR cells. The tumor-targeting molecules bind to target cells in the subject by association to a tumor-associated antigen or a tumor-specific antigen.

The tumor-targeting molecules may be formulated for administration to a subject using techniques known to the skilled artisan. Formulations of the tumor-targeting molecules may include pharmaceutically acceptable excipient(s). Examples of generally used excipients include, without limitation: saline, buffered saline, dextrose, water-for-injection, glycerol, ethanol, and combinations thereof, stabilizing agents, solubilizing agents and surfactants, buffers and preservatives, tonicity agents bulking agents, and lubricating agents.

The tumor-targeting molecules may be administered to a subject using modes and techniques known to the skilled artisan. Exemplary modes include, but are not limited to, intravenous, intraperitoneal, and intratumoral injection. Other modes include, without limitation, intradermal, subcutaneous (s.c., s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect such administration.

Formulations comprising the tumor-targeting molecules are administered to a subject in an amount that is effective for treating and/or prophylaxis of the specific indication or disease. In general, formulations comprising at least about 0.1 mg/kg to about 100 mg/kg body weight of the tumor-targeting molecules are administered to a subject in need of treatment. In most cases, the dosage is from about 1 mg/kg to about 100 mg/kg body weight of the tagged proteins daily, taking into account the routes of administration, symptoms, etc. A physician will determine appropriate dosages to be used.

In one embodiment, the B cell receptor like complex is used for stimulating a T cell-mediated immune response. A T cell-mediated immune response is an immune response that involves the activation of T cells. Activated antigen-specific cytotoxic T cells are able to induce apoptosis in target cells displaying epitopes of foreign antigens on their surface, such as for example cancer cells displaying tumor antigens. In another embodiment, the B cell receptor like complex is used to provide anti-tumor immunity in the mammal. Due to a T cell-mediated immune response the subject will develop an anti-tumor immunity.

The present invention relates to methods of treating a subject having cancer comprising administering to a subject in need of treatment one or more formulations of tumor-targeting molecules, wherein these molecules bind to a cancer cell, and administering one or more therapeutically-effective populations of T-BCR cells, wherein the T-BCR cells bind the tumor-targeting molecules and induce cancer cell death. Another embodiment of the invention relates to methods of treating a subject having cancer comprising administering to a subject in need of treatment one or more therapeutically-effective populations of T-BCR cells, wherein the T-BCR cells bind to a cancer cell, thereby inducing cancer cell death.

Administration frequencies of both formulations comprising T-BCR cells and T-BCR cells in combination with tumor-targeting molecules will vary depending on factors that include the disease being treated, the elements comprising the T-BCR cells and the tumor-targeting molecules, and the modes of administration. Each formulation may be independently administered 4, 3, 2, or once daily, every other day, every third day, every fourth day, every fifth day, every sixth day, once weekly, every eight days, every nine days, every ten days, bi-weekly, monthly and bi-monthly.

The duration of treatment will be based on the disease being treated and will be best determined by the attending physician. However, continuation of treatment is contemplated to last for a number of days, weeks, or months.

The term “cancer” is intended to be broadly interpreted and is defined as disease characterized by the rapid and uncontrolled growth of aberrant cells. Cancer cells can spread locally or through the bloodstream and lymphatic system to other parts of the body. Examples include: carcinoma, including but not limited to adenocarcinoma, squamous cell carcinoma, adenosquamous carcinoma, anaplastic carcinoma, large cell carcinoma, small cell carcinoma, and cancer of the skin, breast, prostate, bladder, vagina, cervix, uterus, ovary, liver, kidney, pancreas, spleen, lung, trachea, bronchi, colon, small intestine, stomach, esophagus, gall bladder; sarcoma, including but not limited to chondrosarcoma, Ewing's sarcoma, malignant hemangioendothelioma, malignant schwannoma, osteosarcoma, soft tissue sarcoma, and cancer of bone, cartilage, fat, muscle, vascular, and hematopoietic tissues; lymphoma and leukemia, including but not limited to mature B cell neoplasms, such as chronic lymphocytic leukimia/small lymphocytic lymphoma, B-cell prolymphocytic leukemia, lymphomas, and plasma cell neoplasms, mature T cell and natural killer (NK) cell neoplasms, such as T cell prolymphocytic leukemia, T cell large granular lymphocytic leukemia, aggressive NK cell leukemia, and adult T cell leukemia/lymphoma, Hodgkin lymphomas, and immunodeficiency-associated lymphoproliferative disorders; germ cell tumors, including but not limited to testicular and ovarian cancer; blastoma, including but not limited to hepatoblastoma, medullobastoma, nephroblastoma, neuroblastoma, pancreatoblastoma, leuropulmonary blastoma and retinoblastoma. The term also encompasses benign tumors.

As used herein, the term “autologous” is meant to refer to any material derived from the same individual to which it is later to be re-introduced into the individual.

An engineered cell or a pharmaceutical composition comprising one or more of said engineered cells disclosed herein can be administered in combination with another anti-tumor agents, including a chemotherapeutic agent, a cytotoxic/antineoplastic agent or an anti-angiogenic agent.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Generation and Functional Characterization of CD20-Specific T Cells using a T-BCR

This example demonstrates the generation of a CD20-specific B cell receptor like complex and its functional expression in human T cells. In particular, a CD20-specific T-BCR comprising a membrane-bound IgG1-isotype antibody against CD20 and a CD79αβ heterodimer carrying a CD28/CD3ζ signaling domain on both protein-chains was designed and functionally evaluated.

Design of the T-BCR Transgene Cassettes

The T-BCR complex comprises a membrane-bound antibody and a CD79αβ heterodimer carrying a CD28/CD3 signaling domain on both protein-chains. In order to express a given antibody bound to the membrane, any potential secretion signal (CH-S) in the Immunoglobulin (Ig) heavy chain was replaced with the trans-membrane domain (M-region) corresponding to the isotype of the utilized antibody. Both Ig heavy- and Ig light-chains are modified with a leader-peptide. CH-S, M-regions and leader peptides can be retrieved for example from the IMGT database (http://www.imgt.org/). Sequences of the CD79αβ heterodimer can be retrieved from protein-databases such as the NCBI protein database (http://www.ncbi.nlm.nih.gov/protein).

Intracellular signaling domains controlling T cell activity are fused to the CD79α, CD79β, or both molecules after the respective trans-membrane-domain.

In order to achieve bi- or multicistronic gene expression for example of the CD79αβ proteins or the Ig heavy and light chain, previously described gene elements such as internal ribosomal entry side (IRES) or 2A peptide sequences can be used.

In the present example, a CD28/CD3 zeta signaling domain was fused to both CD79α and CD79β. Both CD79α and CD79β were fused with a P2A peptide sequence. The resulting protein sequence of this complex is depicted in FIG. 2 and in SEQ ID NO.: 5:

The variable segments of the CD20-specific antibody Rituximab were obtained from public databases and fused to Ig-constant domains.

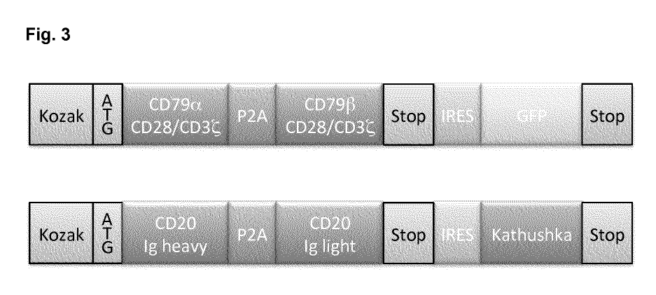

Protein-sequences were expressed from codon-optimized gene-sequences carrying all the elements necessary for expression (e.g. Kozak-sequences). In order to ease the assessment of transgene integration into the genome of T cells, retroviral vectors additionally carrying genes encoding the eGFP and Katushka fluorochromes were used. These fluorochromes were expressed from an internal ribosomal entry side (IRES). A schematic overview of the transgenes is provided in FIG. 3.

Expression of the T-BCR Complex in T Cells

Codon-optimized synthetic genes of the T-BCR components as discussed above were obtained from commercial suppliers and cloned into a pMP71 retroviral expression vector suitable for transduction of T cells.

For retrovirus production, suitable packaging cells (FLYRD18 cells) were plated into 10-cm dishes at 1.2×10⁶ cells per dish. After 24 h, cells were transfected with 10 μg retroviral vector DNA using a transfection reagent (e.g. FuGENE 9 Promega or X-treme gene 9 Roche Diagnostics).

In order to generate CD20-specific T cells both constructs comprising the T-BCR as shown in FIG. 3 were introduced into human donor T cells. Primary human T cells were isolated and activated from human peripheral blood mononuclear cells (PBMCs). In particular, human T cell expander beads (Life Technologies) were used to select CD3⁺ cells from PBMC material and activate 1.5×10⁶ CD3⁺ cells per well in a 24-well plate with 100 IU ml⁻¹ rh-IL-2 and 5 ng ml⁻¹ rh-IL-15 (Peprotech). After 48 h, 0.2×10⁶ to 0.5×10⁶ activated CD3⁺ cells were resuspended in 0.5 ml harvested retroviral supernatant and 0.5 ml medium supplemented with rh-IL-2 (100 IU ml⁻¹ final) and rh-IL-15 (5 ng ml⁻¹ final) and transferred to Retronectin (Takara)-coated plates. Plates were centrifuged for 90 minutes at 430 g.

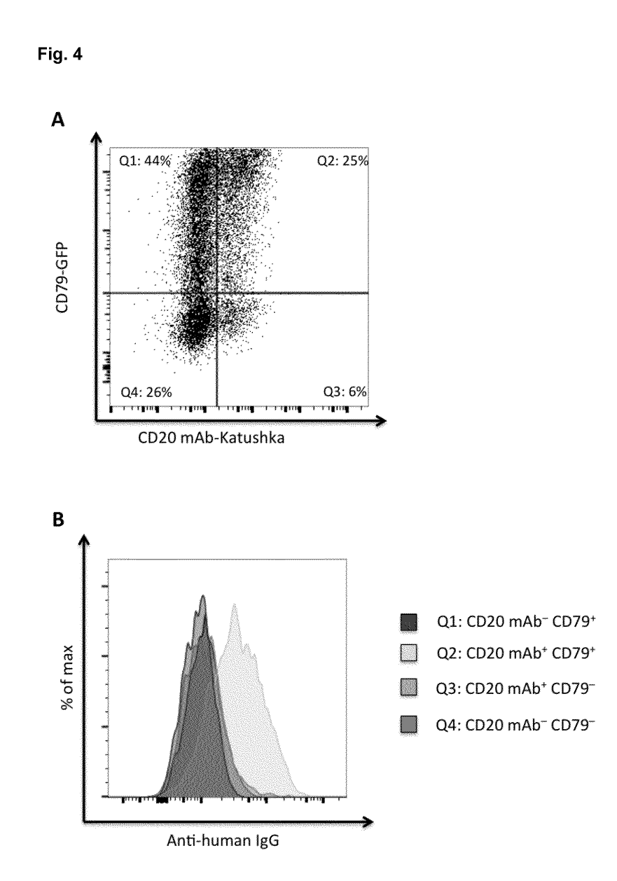

Transduction efficiency of T cells was determined by flow-cytometry at 72 h. In particular, expression levels of the membrane-bound CD20 antibody and the CD79αβ heterodimer were measured by assessment of Katushka and eGFP expression levels respectively by flow cytometry. As shown in FIGS. 4A and B, transduction efficiency of T cells with the CD-20 specific T-BCR complex was 26% as represented by the fraction of human T cells expressing both eGFP (CD79αβ heterodimer) and Katushka (CD20).

Functional Characterization of T Cells Expressing the CD20-Specific T-BCR Complex

Subsequently, the capacity of the CD20 T-BCR expressing T cells to recognize human B cells lines and their subsequent activation was tested. In general, activation of T cells expressing the T-BCR complex can be measured by IFN-γ (or comparable cytokines) production after stimulation with the cognate antigen (e.g. CD20).

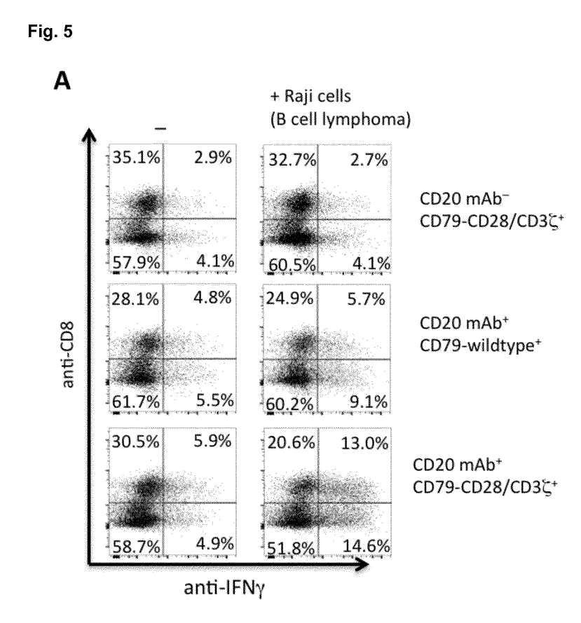

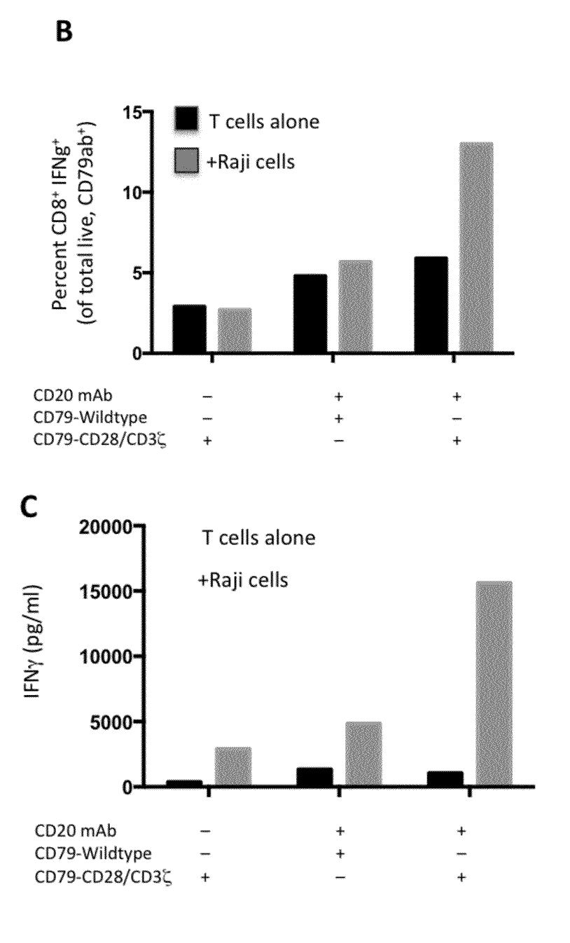

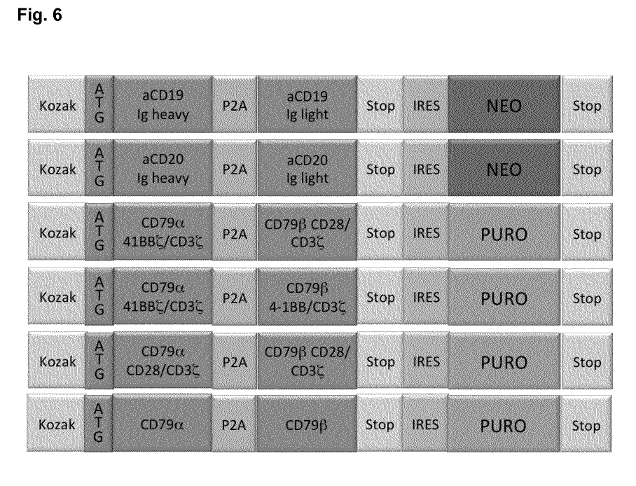

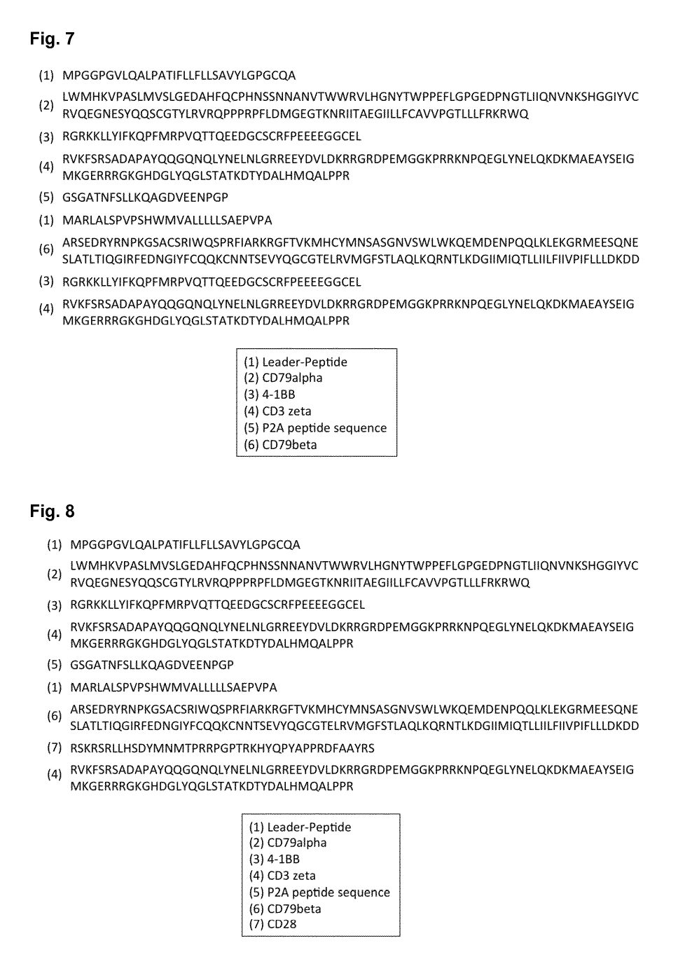

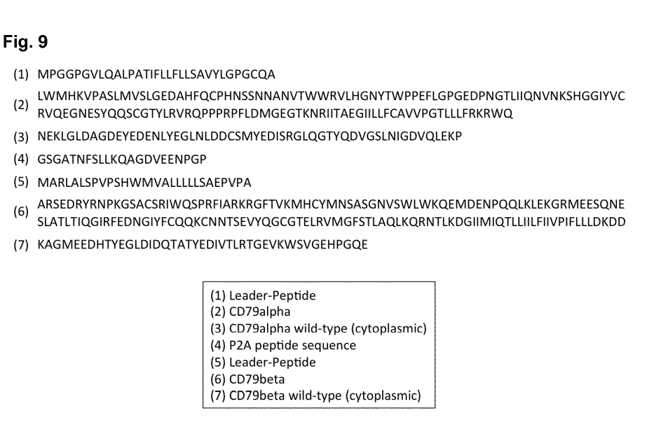

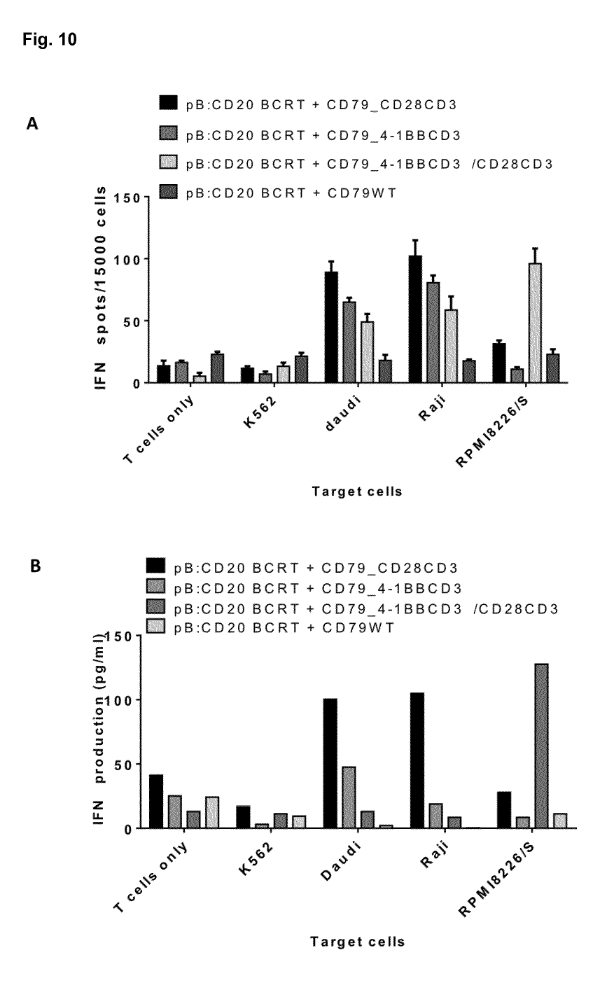

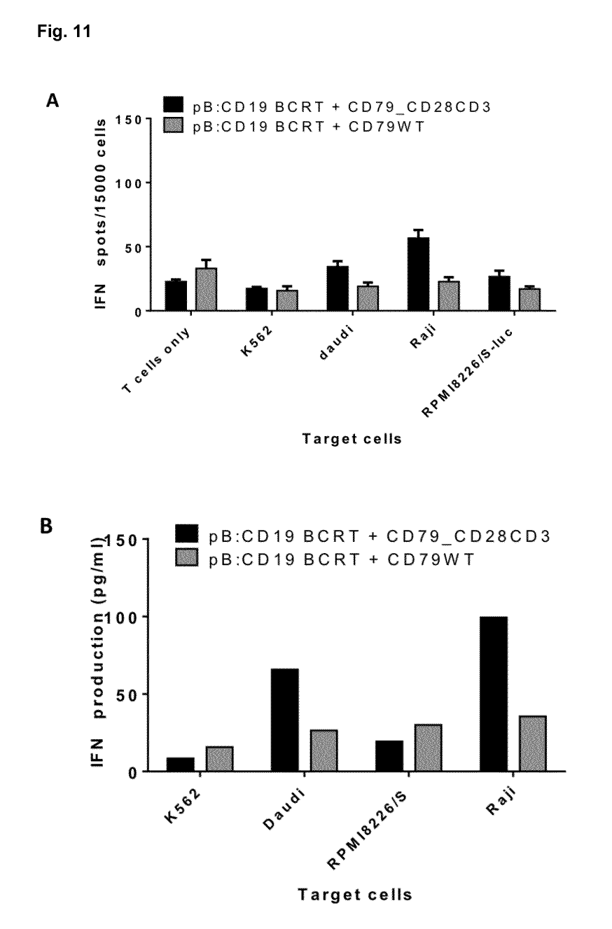

1×10⁵ T-BCR-transduced T cells were incubated with 1×10⁵ Raji cells expressing the cognate antigen of the T-BCR (in this example CD20⁺ cells together with a CD20 T-BCR). After 16 h incubation in the presence of 1 μl ml⁻¹ Golgiplug (BD Biosciences) at 37° C., cells were washed and stained with antibodies against CD3, CD8 (both BD Biosciences) and a suitable life/dead dye (IR dye, Life Technologies). Intracellular levels of IFN-γ were subsequently determined on single-cell basis by flow cytometry using the Cytofix/Cytoperm kit (BD Biosciences) and an antibody against IFN-γ (BD Biosciences), according to the manufacturers guidelines. The data were normalized by correction of percentage of IFN-γ⁺CD8⁺ T cells with the frequency of T-BCR Td CD8⁺ T cells as measured by antibodies against human CD79α, CD79β and the Ig heavy or light chain (all from BD Biosciences). As represented in FIGS. 5A and B, incubation of the CD20-specific T-BCR expressing T cells (CD20⁺ and CD79-CD28/CD3zeta⁺) with Raji cells resulted in a significant increase in intracellular IFN-γ levels in the T cells, as compared to T cells expressing CD20 with only

CD79 wildtype or T cells expressing only CD79-CD28/CD3zeta without expression of the CD20 antibody.

Further, IFN-γ levels in the culture supernatants of T cells transduced with different variants of a T-BCR and stimulated with Raji cells were measured using Cytometric Beas array assay (Life Technologies). As depicted in FIG. 5C, CD20-specific T-BCR transduced T cells secreted significantly more IFN-γ as compared to T cells expressing CD20 with only CD79 wildtype or T cells expressing only CD79-CD28/CD3zeta without expression of the CD20 antibody CD20 mAb or CD79-CD28/CD3zeta only.

Altogether, these date indicate that the CD20-specific T-BCR complex is functionally expressed in human T cells.

### Example 2

### Design and Functional Characterization of Different T-BCR Complexes

In Example 2, the functionality of different T-BCR complexes comprising various extracellular antigen recognition domains and various co-stimulatory molecules in the signaling region was be evaluated. In particular, the design and functionality of various T-BCR complexes comprising CD20 or CD19 extracellular antigen recognition domains and CD28 and/or 4-1BB as co-stimulatory molecules in the signaling region are evaluated.

For all assays described below, effector cells, target cells and negative control cells are used. Effector cells generally refer to the T cells transduced with the T-BCR complex transgenes. Target cells are generally tumor cells naturally expressing the target (antigen) to which the T-BCR complex is directed. Negative control cells are generally cells that do not express the target (antigen) to which the T-BCR complex can bind.

Cells and Cell Lines

Daudi, K562, Raij and Phoenix-Ampho cells were obtained from the American Type Culture Collection. RPM18226/S-luc (RPMI-Luc) was kindly provided by Anton Martens, (University Medical Center Utrecht, The Netherlands), Phoenix-ampho cells were cultured in DMEM+1% Pen/Strep (Invitrogen)+10% FCS (Bodinco), all other cell lines in RPMI+1% Pen/Strep+10% FCS. PBMCs were isolated from buffy coats obtained from the Sanquin Blood Bank (Amsterdam, The Netherlands) or from the Institute for Transfusion Medicine and Immunohematology, Frankfurt, Germany.

Construction of T-BCR Gene Cassettes in Retroviral Vectors

The various constructs for the different T-BCR complexes that are evaluated in this example are presented in FIG. 6. Corresponding protein sequences of the different constructs are represented in FIGS. 2, 7, 8 and 9, and in SEQ ID NOs 5 to 13.

Retro Viral Transduction of T-Cells

T-BCR and CD79 constructs were transduced into apT-cells as previously described (3). In brief, packaging cells (phoenix-ampho) were transfected using FugeneHD reagent (Promega, Madison, Wis., USA) with helper constructs gag-pol (pHIT60), env (pCOLT-GALV) (4). In addition, for the BCR-T cells two retroviral vectors containing either CD79α-P2A-CD79β-IRES-neomycine or Igheavy-P2A-Iglight-IRES-puromycine (pBullet vector), or CD79α-P2A-CD79β-IRES-GFP or IgGheavy-P2A-IgGlight-IRES-Katusha (pMP71) were added. Human PBMC were pre-activated with αCD3 (30 ng/ml) (Orthoclone OKT®3, Janssen-Cilag, Tilburg, The Netherlands) and IL-2 (50 IU/ml) (Proleukin®, Novartis, Arnhem, The Netherlands) and transduced twice with viral supernatant within 48 hours in the presence of 50 IU/ml IL-2 and 6 μg/ml polybrene (Sigma-Aldrich, Zwijndrecht, The Netherlands). Transduced T-cells were expanded by stimulation with αCD3/CD28 Dynabeads (0.5×10⁶ beads/10⁶ cells) (Life Technologies, Carlsbad, Calif., USA) and IL-2 (50 IU/ml) and in case of pBullet retroviral system selected with 800 μg/ml geneticin (Gibco, Karlsruhe, Germany) and 5 μg/ml puromycin (Sigma-Aldrich). Next, T-BCR-transduced T-cells were expanded based on a previously described rapid expansion protocol (REP).

Flow Cytometry

To evaluate the transduction efficiency of the T cells with the various constructs, flow cytometry analysis was performed. Antibodies used for flow cytometry include: anti-CD4-FITC (clone RPA-T4), anti-CD8-PerCP.Cy5.5 (clone RPA-T8, both BD Biosciences, San Jose, USA) and anti-CD79β-PE (clone ZL9-3, Santa Cruz). Expression of IgG was analyzed by staining with Protein L-biotin followed by streptavidin-PE or Goat-anti-Human-IgG-PE (Jackson ImmunoResearch Laboratories, West Grove, Pa., USA). Samples were analyzed on a FACS LSRII or FACS Canto using FACSdiva software (BD Biosciences).

Cytotoxicity Assays

To evaluate the potential cytotoxic effect of the transduced T cells, different cytotoxicity assays will be performed.

In the ⁵¹Chromium-release assay for cell-mediated cytotoxicity, target cells will be labeled overnight with 100 μCu ⁵¹Cr and incubated for 4-5 h with the transduced T cells in 5 different effector-to-target-ratios (E:T), varying between 30:1 and 0.3:1. Percentage of specific lysis will be calculated as follows: (experimental cpm−basal cpm)/(maximal cpm−basal cpm)×100 with maximal lysis determined in the presence of 5% triton and basal lysis in the absence of effector cells.

In another cytotoxicity assay, negative control cells are suspended in medium at a concentration 1.5*10⁶ cells/mL, and the fluorescent dye 5-(and-6)-(((4-chloromethyl)benzoyl)amino) tetramethylrhodamine (CMTMR) (Invitrogen) is added at a concentration of 5 μM. The cells are mixed and then incubated at 37° C. for 30 minutes. The cells were then washed and suspended in cytotoxicity medium. Next, the negative control cells are incubated at 37° C. for 60 minutes. The cells are then washed twice and suspended in cytotoxicity medium.

Target cells are suspended in PBS+0.1% BSA at 1*10⁶ cells/mL. The fluorescent dye carboxyfluorescein diacetate succinimidyl ester (CFSE) (Invitrogen) is added to this cell suspension at a concentration of 1 μM. The cells are incubated 10 minutes at 37° C. After the incubation, the labeling reaction was stopped by adding a volume of FBS that is equal to the volume of cell suspension and the cells are incubated for 2 minutes at room temperature. The cells are washed and suspended in cytotoxcity medium.

Subsequently, effector engineered T cells are washed and suspended at 5*10⁶ cells/mL in cytotoxicity medium. In all experiments, the cytotoxicity of effector T cells that are transduced with the T-BCR constructs is compared to the cytotoxicity of negative control effector T cells from the same patient that were transduced with the negative control BCRT or were not transduced. For effector T cells and negative control effector T cells, cultures are set up in sterile 5 mL test tubes (BD Biosciences) in duplicate at the following T cell:target cell ratios: 10:1, 3:1, and 1:1. The target cells are always 50,000 PBMC from a CLL patient. Each culture also contains 50,000 negative control cells. In addition, tubes are set up that contain only target cells plus negative control cells. The cultures are incubated for 4 hours at 37° C. Immediately after the incubation, 7AAD (7-aminoactinomycin D) (BD Pharmingen) is added as recommended by the manufacturer, and flow cytometry acquisition was performed with a BD FacsCanto II (BD Biosciences). Analysis was performed with FlowJo (Treestar, Inc. Ashland, Oreg.). Analysis is gated on 7AAD-negative (live) cells, and the percentages of live target cells and live negative control cells are determined for each T cell+target cell culture. For each T cell+target cell culture, the percent survival of target cells is determined by dividing the percent live target cells by the percent live negative control cells. The corrected percent survival of target cells calculated by dividing the percent survival of target cells in each T cell+target cell culture by the ratio of the percent target cells:percent negative control cells in tubes containing only target cells and negative control cells without any effector T cells. This correction is necessary to account for variation in the starting cell numbers and for spontaneous target cell death. Cytotoxicity was calculated as the percent cytotoxicity of target cells=100-corrected percent survival of target cells. For all effector:target ratios, the cytotoxicity was determined in duplicate and the results were averaged.

Proliferation Assay

Proliferation of engineered T cells after exposure to target cells is determined by carboxyfluorescein succinimidyl ester dilution assays (Hudecek M, Lupo-Stanghellini M T, Kosasih P L, Sommermeyer D, Jensen M C, Rader C et al. Receptor affinity and extracellular domain modifications affect tumor recognition by ROR1-specific chimeric antigen receptor T cells. Clin Cancer Res 2013; 19: 3153-3164)

One week posttransduction, control and engineered T lymphocytes will be labeled with 1.5 μmol/L carboxyfluorescein diacetate succinimidyl ester (CFSE; Invitrogen) and plated with irradiated tumor targets (CD19 positive and negative lines: NALM-6 and CEM) at an effector-to-target (E:T) ratio of 5:1. CFSE dilution will be measured on CD4⁺ and CD8⁺ T cells by flow cytometry on day 4 of coculture.

T Cell Activation Assays

a) ELISPOT

Activation of the transduced T cells expressing the different T-BCR complexes after contact with effector cells were evaluated by measurement of secreted IFN-γ levels. IFN-γ ELISPOT was performed using anti-hu IFN-γ mAb1-D1K (I) and mAb7-B6-1 (II) (Mabtech-Hamburg, Germany) using the ELISA-ready-go! Kit (eBioscience, San Diego, Calif., USA) following the manufacturer's recommended procedure. Effector and target cells (E:T 1:1) were incubated for 24 h at 37° C. before supernatant was harvested and analyzed for the production of IFN-γ. In general, the presence of T cell effector cytokines (e.g. IFN-γ, IL-2, TNF-α) are quantified by

ELISA, Cytokine bead array or comparable methods.

b) CD107

Further, intracellular IFN-γ levels in the T cells will be evaluated using a CD107 assay. Transduced T cells will be incubated with effector cells in the presence of a CD107a-PE antibody and Golgistop for 4-5 h at 37° C. Next, cells will be harvested and stained with anti-CD8 antibodies and analyzed by flow cytometry. CD107 expression will demonstrate activation in the effector population incubated with antigen-expressing tumor cells while control cells and effector cells incubated with an antigen-negative target will have low or no CD107 expression.

c) ELISA

In addition ELISA assays were utilized to determine T cell activation. Tumor target cells were washed and suspended at 1×10⁶ cells per mL in T cell media without IL-2. One-hundred-thousand target cells of each target cell type were added to each of two wells of a 96 well round bottom plate (Corning). Effector T cell cultures (BCR-T and Control T cells) were washed and suspended at 1×10⁶ cells per mL in T cell media without IL-2. One-hundred-thousand effector T cells were combined with target cells in the indicated wells of the 96-well plate. As a control, wells containing T cells alone were prepared. The plates were incubated at 37° C. for 18-20 hours. Following the incubation, an IFNγ ELISA assay was be performed using standard methods (Pierce, Rockford, Ill.).

Statistical Analyses

Differences are analyzed using indicated statistical tests in GraphPad Prism (GraphPad Software Inc., La Jolla, Calif., USA).

Results

Primary T cells were retrovirally engineered with pB:CD20mAb_NEO in combination with pB:CD79_CD28CD3ζ_PURO, pB:CD79_4-1BBCD3ζ_PURO, pB:CD79CD28CD3ζ/4-1BBCD3ζ_PURO or pB:CD79WT_PURO. Following introduction of transgenes T cells were cultured in the presence of geneticin and puromycin and expanded using a rapid expansion protocol (REP). Two weeks after expansion of the engineered T cells, they were co-cultured with different type of tumor cells (K562 (Chronic Myeloid Leukemia; CD19⁻ CD20⁻), Daudi (B cell lymphoma; CD19⁺⁺, CD20⁺⁺), Raji (B cell lymphoma; CD19⁺⁺, CD20⁺⁺) and RPM18226/S (Multiple Myeloma; CD19⁻, CD20^{-/+})) for 24 hours at 37° C. and IFNγ secretion was measured by ELISPOT or ELISA (FIGS. 10A and B). Co-culture of the engineered T cells with the Daudi cells and the Raji cells resulted in an increased IFNγ secretion in the engineered T cells that were engineered to express the CD20-BCRT-CD79/CD28/CD3, CD20-BCRT-CD79/4-1BB/CD3, CD20-BCRT-CD79/4-1 BB/CD28/CD3 complexes, but not in the T cells that were engineered to express the CD20-BCRT CD79 wildtype complex. Importantly, the inclusion of multiple different co-stimulatory domains in the T-BCR complex resulted in T cell activation. In addition, target cells with low CD20 expression (RMP18226/S) were recognized by T cells expressing the T-BCR complex that comprised a combination of CD28 and 4-1 BB. This may indicate that the combination of different co-stimulatory domains reduces the activation threshold of the T cells and make them more sensitive.

Further, primary T cells were retrovirally engineered with pB:CD19mAb_NEO in combination with pB:CD79_CD28CD3ζ_PURO or pB:CD79WT_PURO. Following introduction of transgenes T cells were cultured in the presence of geneticin and puromycin and expanded using a rapid expansion protocol (REP). After 2 weeks of expansion T cells were co-cultured with the above-listed tumor cells for 24 hours at 37° C. and !My secretion was measured by ELISPOT or ELISA (FIGS. 11A and B). Co-culture of the engineered T cells with the Daudi cells and the Raji cells resulted in an increased IFNγ secretion in the engineered T cells that were engineered to express the CD19-BCRT-CD79/CD28/CD3 complex, but not in the T cells that were engineered to express the CD19-BCRT CD79 wildtype complex.

### Example 3

### In Vivo evaluation of CD19- or CD20 Specific T-BCR T Cells

The therapeutic potential of CD19- or CD20-specific T-BCR T cells will be evaluated in a Daudi or Raji B cell lymphoma mouse model.

The RAG2^{-/-}/γc^{-/-}-BALB/C mice or NOD/SCID mice are bred and housed in the specific pathogen-free (SPF) breeding unit. Experiments will be conducted according to Institutional Guidelines. 10⁷ CD19-specific T-BCR transduced, CD19- or CD20-specific T-BCR transduced or Mock transduced T cells will be i.v. injected simultaneously with 0.5×10⁶ Daudi-Luc or Raji-FFLuc B lymphoma cells via the tail vein. Alternatively, CD19- or CD20-specific T-BCR transduced or Mock transduced T cells will be i.v. injected on day 2, 5 or 10 after tumor cell injection. Optionally, mice receive a second injection of transduced T cells 5 days after the first injection with transduced T cells. Mice receive 0.6×10⁶ IU of IL-2 in IFA s.c. on day 1 and every 21 days till the end of the experiment. Tumors are visualized in vivo by bioluminescent imaging. To this end, mice will be anesthetized by isoflurane followed by an i.p. injection (100 μl) of 25 mg/ml Beetle Luciferin (Promega). Bioluminescence images will be acquired by using a third generation cooled GaAs intensified charge-coupled device camera, controlled by the Photo Vision software and analyzed with M3Vision software (all from Photon Imager; Biospace Laboratory, Paris, France). Samples from blood, bone marrow, spleen and liver are collected 12 hours after the second injection and the presence of transferred T cells and tumor cells is evaluated by flow cytometry.

### Example 4

### Clinical Grade Expansion of CD19- or CD20-Specific T-BCR T Cells

In order to generate a large number of engineered T cells, the cells were induced to proliferate using a rapid expansion protocol (REP). Prior to being used in REPs, T cells had been started in culture with OKT3, anti-CD28 and IL-2 and transduced on the second and third days after the initiation of culture as detailed above. The cells were cultured in a 75 cm² flask at 37° C. and 5% CO₂. The cells were counted and suspended at a concentration of 0.5×10⁶ cells/mL in fresh T cell medium with 300 IU/mL of IL-2 every two days for the remainder of the time they were kept in culture.

### Example 5

### Clinical Trial of T-BCR Cells

To determine clinical efficacy and safety of T-BCR cell, CD19- specific T-BCR T cells will be evaluated in a clinical trial setting. A subject in need thereof, with a CD19⁺ cancer will be enrolled into a phase I dose escalation trial.

Anti-CD19 T-BCR engineered cells will be produced by adding the anti-CD3 monoclonal antibody (OKT3) directly to whole peripheral-blood mononuclear cells (PBMCs), obtained from the subject in need thereof, suspended in culture medium containing interleukin-2 (IL-2). Anti-CD19 T-BCR cells were produced by activating peripheral-blood mononuclear cells (PBMCs) with anti-CD3 antibody OKT3 on day 0 and retrovirally transducing the T cells on day 2, as described in Example 1. A disposable WAVE Bioreactor system will be utilized to transduce and expand, in IL-2, transduced T-BCR cells to large numbers. T-BCR T cells will be dosed as a number of CD3⁺ T-BCR-positive cells/kg bodyweight. The percentage of T-BCR-positive T cells will be determined by flow cytometry and used to calculate the total number of cells to infuse to achieve the target dose in the subject that is being treated. Long-term eradication of normal CD19⁺ B cells from subjects receiving infusions of anti-CD19 T-BCR cells demonstrates the potent antigen-specific activity of the T-BCR cells.

Safety assessments will be performed weekly while the patients are receiving therapy, and then every 4 weeks for an additional 12 weeks. Hematologic toxicity will be graded according to the IWCLL 2008 criteria and during the first cycle of therapy and will be defined as any 1 of the following adverse events with a possible, probable, or unknown relationship to therapy: >grade 3 tumor lysis syndrome or grade 3 tumor lysis syndrome requiring dialysis, grade 4 fatigue lasting for days, any other grade 3 nonhematologic toxicity (excluding nausea, vomiting, electrolyte abnormality, or liver function abnormality in the absence of 3 days of maximal antiemetic/electrolyte replacement therapy), grade 4 neutropenia (ANC <0.5×10⁹) lasting for ≧7 days in patients with pretreatment ANC >1×10⁹, or any other grade hematologic toxicities lasting for greater than 3 days excluding lymphocytopenia.

Responses will be determined according to IWCLL 2008 guidelines, which incorporate physical examination and clinical laboratory data as well as computed tomography (CT) scan data for CLL, and per the 2007 International Working Group Response Criteria for SLL. Responses will be evaluated on cycle 2, day 1; end of cycle 2; and 4, 8, and 12 weeks after the end of cycle 2.

1. A B cell receptor like complex comprising:
an extracellular antigen recognition domain,
a trans-membrane domain,
a CD79 protein or a functional equivalent thereof, and
a signaling region that controls T cell activation;
wherein the extracellular antigen recognition domain and the trans-membrane domain are derived from the same human or humanized B cell receptor protein, and
wherein the signaling region comprises a T cell signaling domain in combination with a co-stimulatory domain, and wherein the signaling region is fused to the CD79 protein.

2. An isolated nucleic acid encoding the B cell receptor like complex or a fragment of the B cell receptor like complex according to claim 1.

3. The B cell receptor like complex according to claim 1, wherein the CD79 protein consists of a CD79α protein, a CD79β protein, a CD79α homodimer, a CD79β homodimer, a CD79αβ heterodimer, or any functional equivalents thereof.

4. The B cell receptor like complex according to claim 1, wherein the T cell signaling domain, the co-stimulatory domain or both are fused to one or both monomers of the CD79 protein.

5. The B cell receptor like complex according to claim 1, wherein the extracellular antigen recognition domain and the trans-membrane domain form a single human or humanized B cell receptor protein.

6. The B cell receptor like complex according to claim 1, wherein the extracellular antigen recognition domain binds to a surface antigen.

7. The B cell receptor like complex according to claim 1, wherein the extracellular antigen recognition domain binds to a universal epitope expressed on a targeting molecule.

8. (canceled)

9. The B cell receptor like complex of claim 7, wherein the targeting molecule is a protein scaffold selected from the group consisting of scFv molecules, Darpin molecules, Nanobody molecules, Alphabody molecules, Centyrin molecules, Affibody molecules, and heavy chain only antibodies.

10. The B cell receptor like complex of claim 7, wherein the targeting molecule binds to a surface antigen associated with a solid or hematologic tumor.

11. (canceled)

12. The B cell receptor like complex of claim 1, wherein the T cell signaling domain contains one or more ITAM motifs leading to T cell activation.

13. The B cell receptor like complex of claim 1, wherein the co-stimulatory domain comprises one or more fragments of the intracellular domain of a co-stimulatory molecule selected from CD27, CD28, 4-1BB, OX40, CD30, CD40L, ICOS, lymphocyte function-associated antigen (LFA-1), CD2, CD7, NKG2C, GITR, CD137, HVEM, TIM1, Galectin-9, a ligand that specifically binds with CD83, and any combination thereof.

14. An engineered cell comprising the B cell receptor like complex according to claim 1.

15. The engineered cell of claim 14, wherein the cell is a T cell.

16. (canceled)

17. A vector comprising the isolated nucleic acid according to claim 2.

18. An engineered cell comprising the vector according to claim 17.

19. A process for generating an engineered cell, wherein the vector according to claim 17 is introduced into a cell.

20. The process for generating an engineered cell according to claim 19, wherein the vector is introduced into the cell by viral gene delivery technology or by non-viral gene delivery technology.

21. (canceled)

22. A pharmaceutical composition comprising the engineered cell according to claim 14.

23.-24. (canceled)

25. A method for stimulating a T cell-mediated immune response to a target cell population or tissue in a mammal, the method comprising administering to a mammal an effective number of engineered cells according to claim 14, thereby stimulating a T cell-mediated immune response in the mammal.

26. A method of providing an anti-tumor immunity in a mammal, the method comprising administering to a mammal an effective number of engineered cells according to claim 14, thereby providing anti-tumor immunity in the mammal.

27. A method of treating a mammal having a disease, disorder or condition associated with an aberrant expression of an antigen, the method comprising administering to a mammal an effective number of engineered cells according to claim 14, thereby treating the mammal.
